SEQUENCE LISTING (2 sequences)

SEQ ID NO: 1 - DNA, 42 bp, Artificial Sequence
  Description of Artificial Sequence Synthetic primer
cgccagggttttcccagtcacgaaccaatctcagctgaaagc

SEQ ID NO: 2 - DNA, 43 bp, Artificial Sequence
  Description of Artificial Sequence Synthetic primer
gagccactatcgactacggcatcaatatctctcctggtggctg

(19) United States
(12) Patent Application Publication
(10) Pub. No.: US 2019/0062416 A1
(43) Pub. Date: Feb. 28, 2019

(54) METHOD OF TREATING PRIMARY FOCAL SEGMENTAL GLOMERULOSCLEROSIS

(71) Applicant: GENZYME CORPORATION, Cambridge, MA (US)

(72) Inventors: Steven R. Ledbetter, Westborough, MA (US); Sara Engstrand, Madison, WI (US); Patrick Finn, Bridgewater, NJ (US); Julie Lin, Milton, MA (US); Robert John Pomponio, Bridgewater, NJ (US)

(73) Assignee: GENZYME CORPORATION, Cambridge, MA (US)

(21) Appl. No.: 15/737,069

(22) PCT Filed: Jun. 17, 2016
(86) PCT No.: PCT/US16/37963

§ 371 Date: Dec. 15, 2017

Related U.S. Application Data
(60) Provisional application No. 62/182,102, filed on Jun. 19, 2015.

(51) Int. Cl. C07K 16/22; A61K 9/19; A61P 13/12

(52) U.S. Cl. CPC C07K 16/22; A61K 2039/54; A61P 13/12; A61K 9/19

(57) ABSTRACT

A method of treating primary focal segmental glomerulosclerosis in a patient having an APOL1 variant, comprising intravenously administering a TGFβ antagonist to the patient.

### CROSS REFERENCE TO RELATED APPLICATION

This application is a National Stage under 35 U.S.C. § 371 of International Application No. PCT/US2016/037963, filed Jun. 17, 2016, which claims the benefit of priority from U.S. Provisional Application No. 62/182,102, filed Jun. 19, 2015. The foregoing related applications, in their entirety, are incorporated herein by reference.

U.S. Pat. No. 7,723,486, U.S. Pat. No. 8,383,780, and U.S. Pat. No. 8,591,901, in their entirety, are also each incorporated herein by reference.

### FIELD OF THE INVENTION

The present invention relates to a method of treating primary focal segmental glomerulosclerosis (primary FSGS) and/or improving one or more symptoms associated with primary FSGS, comprising administering a TGFβ antagonist to a patient in need thereof. In particular, the method relates to administering a pharmaceutical formulation comprising a therapeutically effective amount of fresolimumab, to a patient having primary FSGS, such as a patient having a genetic variant associated with an increased risk in having or developing primary FSGS, for example, an APOL1 genetic variant, so that the patient may derive a benefit therefrom.

### BACKGROUND OF THE INVENTION

Focal segmental glomerulosclerosis (FSGS) is a clinical entity that impacts renal function through progressive fibrosis of the glomerulus. FSGS is not a single disease; rather, it is a heterogeneous clinicopathological process identified in renal biopsies that are generally performed in patients with proteinuria or nephrotic syndrome. Primary (or idiopathic) focal segmental glomerulosclerosis (herein after referred to as “primary FSGS”) is a kidney disease diagnosed based on clinicopathological finding. Primary FSGS has a distinctive histopathological appearance, characterized by hyalinization and sclerosis of a portion of the glomerular tuft, minimal deposition of immune complexes, and effacement of visceral epithelial cell (podocyte) foot processes. The majority of FSGS cases are characterized by progressive renal fibrosis and steady deterioration in kidney function.

Focal segmental glomerulosclerosis classically presents with nephrotic syndrome: proteinuria ≥3.0 g/24 hours associated with signs/symptoms including hypoalbuminemia, edema and hyperlipidemia. There are many specific causes of nephrotic syndrome, including kidney diseases such as minimal-change nephropathy, focal glomerulosclerosis, and membranous nephropathy. Nephrotic syndrome can also result from systemic diseases that affect other organs in addition to the kidneys, such as diabetes, amyloidosis, and lupus erythematosus. Notably, nephrotic syndrome may affect adults and children, of both sexes and of any race.

Nephrotic syndrome can be primary, being a disease specific to the kidneys, or it can be secondary, being a renal manifestation of a systemic general illness. In all cases, injury to glomeruli is an essential feature. Primary causes of nephrotic syndrome include the following, in approximate order of frequency: minimal-change nephropathy, focal glomerulosclerosis, membranous nephropathy, and hereditary nephropathies. Secondary causes include the following, in approximate order of frequency: diabetes mellitus, lupus erythematosus, amyloidosis and paraproteinemias, viral infections (eg, hepatitis B, hepatitis C, human immunodeficiency virus [HIV]), and preeclampsia.

Primary FSGS is a progressive disease which has a profound impact on quality of life, morbidity and mortality. Diagnosis of primary FSGS is confirmed by renal biopsy which demonstrates glomerular scarring in a focal (involving some but not all glomeruli) and segmental (involving portions of any single glomeruli) pattern. Patients may have profound edema that may lead to retention of liters of fluid impacting mobility and daily activities. If left untreated, primary FSGS spontaneously remits in only a minority (<5%) of patients and typically leads to a relatively rapid decline in renal function resulting in End Stage Renal Disease (herein after referred to as “ESRD”) and need for dialysis or transplant. Renal disease progression brings with it co-morbidities such as hypertension and cardiovascular risk that are further amplified by the hyperlipidemia seen in patients with nephrotic syndrome.

One of the most important prognostic factors for rapid progression to ESRD is the degree of proteinuria. Proteinuria can occur in various forms and at different levels of severity. For example, proteinuria can be classified on the basis of the amount of protein excreted (nephrotic or non-nephrotic), the type of protein excreted (albuminuria or low molecular weight proteinuria), or the underlying pathological damage (glomerular vs non-glomerular).

In non-nephrotic proteinuria, the amount of proteinuria is <3.0 g/24 h and is persistent. These patients require close follow-up and may need a kidney biopsy if they have abnormal urine microscopy results and/or impairment of kidney function. Nephrotic-range proteinuria is defined as ≥3.0 g/24 h of proteinuria excretion rate or ≥3.0 g/gm creatinine on a spot urine protein-to-creatinine ratio. This finding denotes significant glomerular disease and requires a kidney biopsy for diagnosis and management. As a comparison, normal urinary protein excretion is generally considered to be approximately <150 mg/24 hour. For example, in relation to the protein albumin, the daily albumin excretion in a normal person is generally considered to be approximately <30 mg./24 hour.

The presence of nephrotic range proteinuria is associated with poor outcomes and approximately 50% of patients reaching ESRD over 6 to 8 years, while approximately 20% of patients having non-nephrotic proteinuria reach ESRD in about 10 years. More severe proteinuria (>10 g/24 hours) is associated with a more malignant course such that the majority of patients progress to ESRD over 3 years (Korbet S., “Clinical picture and outcome of primary focal segmental glomerulosclerosis,” Nephrol Dial Transplant., 1999; 14:68-73). Additionally, patients who progress to ESRD and receive a renal transplant are at high risk for developing recurrent FSGS in the transplanted graft, with consequences of massive proteinuria and graft failure.

From a therapeutic perspective, nephrotic syndrome may be classified as steroid sensitive, steroid resistant, steroid dependent, or frequently relapsing. Remission of proteinuria is often used as a factor to predict delayed progression to ESRD (Korbet S., Nephrol Dial Transplant., 1999). Currently, there are no approved therapies in steroid-resistant patients. In the absence of approved therapies, multiple therapies have been attempted. For example, immunosuppressive therapies (e.g., Cyclophosphamide and Cyclosporine A) have been used in an attempt to induce remissions with the goal of ameliorating renal functional decline and morbidity associated with nephrosis and renal insufficiency—though none have demonstrated a favorable benefit-risk ratio. Although not a labeled indication, high dose corticosteroids (e.g., Prednisone) have also been commonly used for this purpose but are typically effective in only a relatively small percentage of patients (Korbet S M., “Angiotensin antagonists and steroids in the treatment of focal segmental glomerulosclerosis,” Semin Nephrol., 2003; 23:219-28). For instance, while prolonged treatment with high dose glucocorticosteroids has been associated with a higher remission rate, it also suffers from more frequent complications, including hypertension, hyperglycemia, infection risk, swelling and/or weight gain. To reduce the associated edema, diuretics have often been administered. For the reduction of proteinuria levels in both nephrotic range proteinuria and non-nephrotic proteinuria patients, angiotensin-converting enzyme (ACE) inhibitors and angiotensin II receptor blockers (ARBs) have been administered, and the patients are placed on a low salt diet.

Many of these therapies have also been attempted in patients having FSGS with nephrotic-range proteinuria who are resistant to steroid therapy or intolerant of steroid therapy. In patients with nephrotic syndrome, including patients with steroid-resistant FSGS, alternative immunosuppressive therapies have been tried, such as treating with Cyclosporine A (Brandis, M. et al., “Cyclosporine A for treatment of nephrotic syndromes”, Transplantation Proc. 1988; 20(S4):275-9; Capodicasa, G., et al., “Cyclosporin A in nephrotic syndrome of childhood—a 14 month experience”, Int J Pediatr Nephrol., 1986; 7:69-72; Cattran, D., et al., “A controlled trial of cyclosporine in patients with progressive membranous nephropathy,” Kidney Int. 1995; 47:1130-5; Cattran, D C., et al., “A randomized trial of cyclosporine in patients with steroid-resistant focal segmental glomerulosclerosis,” Kidney Int., 1999; 56:2220-6; Chishti, A S., et al., “Long-term treatment of focal segmental glomerulosclerosis in children with cyclosporine given as a single daily dose,” Am J Kidney Dis., 2001; 38:754-60; Gregory, M J., et al., “Long-term cyclosporine therapy for pediatric nephrotic syndrome: a clinical and histologic analysis,” J Am Soc Nephrol., 1996; 7:543-9; Hino, S., et al., “Follow-up study of children with nephrotic syndrome treated with a long-term moderate dose of cyclosporine,” Am J Kidney Dis., 1998; 31(6):932-9; Meyrier, A., et al., “Remission of idiopathic nephrotic syndrome after treatment with cyclosporin A,” Br Med J., 1986; 292:789-92; Niaudet, P., et al., “Cyclosporin in the treatment of idiopathic nephrotic syndrome in children,” Pediatr Nephrol., 1987; 1:566-73; Niaudet, P., “The French Club of Pediatric Nephrology. Steroid-resistant idiopathic nephrotic syndrome and ciclosporin,” Nephron., 1991; 57:481; Ponticelli, C. et al., “A randomized trial of cyclosporine in steroid-resistant idiopathic nephrotic syndrome,” Kidney Int., 1993; 43:1377-84; Tejani, A., et al., “Cyclosporine A induced remission of relapsing nephrotic syndrome in children,” Kidney Int., 1988; 33:729-34; Lieberman, K V., et al., “New York-New Jersey Pediatric Nephrology Study Group. A randomized double-blind placebo-controlled trial of cyclosporine in steroid-resistant idiopathic focal segmental glomerulosclerosis in children,” J Am Soc Nephrol., 1996; 7:56-63; Walker, R G., et al., “The effect of treatment of corticosteroid-resistant idiopathic (primary) focal and segment hyalinosis and sclerosis (focal glomerulosclerosis) with ciclosporin,” Nephron., 1990; 54:117-21). However, relapse following discontinuation of therapy is frequent and toxicity is a concern. When used for prophylaxis of organ rejection, serious side effects include an increased susceptibility to infection, development of neoplasia particularly lymphoma as well as skin cancers, nephrotoxicity and hypertension. Other therapeutic interventions of past or ongoing investigation include mycophenolate mofetil (Briggs, W A., et al., “Successful mycophenolate mofetil treatment of glomerular disease,” Am J Kidney Dis., 1998; 31:213-7; Choi, M J., et al., “Mycophenolate mofetil treatment for primary glomerular diseases,” Kidney Int., 2002; 61:1098-114; Cattran, D C., et al., “Mycophenolate mofetil in the treatment of focal segmental glomerulosclerosis,” Clin Nephrol., 2004; 62(6):405-11), rituximab (Fernandez-Fernedo, G., et al., “Rituximab treatment of adult patients with steroid-resistant focal segmental glomerulosclerosis,” Clin JAm Soc Nephrol., 2009; 4(8):1317-23) rosiglitazone (Joy, M S., et al., “Phase I trial of rosiglitazone in FSGS: I. Report of the FONT Study Group,” Clin J Am Soc Nephrol., 2009; 4:39-47), sirolimus (rapamycin) (Cho, M E., et al., “Sirolimus therapy of focal segmental glomerulosclerosis is associated with nephrotoxicity,” Am J Kidney Dis., 2007; 49(2):310-17) and cyclophosphamide (Ponticelli, C., et al., “Can prolonged treatment improve the prognosis in adults with focal segmental glomerulosclerosis?” Am J Kidney Dis., 1999; 34(4):618-25). None of these agents are approved for therapy of primary FSGS.

In the majority of primary FSGS patients, however, treatment with steroids and other therapeutic interventions result in only transient improvement in proteinuria and does not alter disease course. This may be especially true in patients with genetic mutations leading to FSGS. Recently, it has been discovered that variation in the apolipoprotein L1 gene locus (herin after referred to as “APOL1”) has been associated with an increased risk in developing FSGS and other related forms of progressive nondiabetic nephropathy, including in populations of recent African ancestry, relative to people of European ancestry (Freedman, Barry I., et al. “Gene-Gene and Gene Environment Interactions in Apolipoprotein L1 Gene-Associated Nephropathy,” Clin. J. Am. Soc. Nephrol., 2014, doi: 10.2215/CJN.01330214). Because of the poor renal prognosis and the lack of a proven treatment for patients with FSGS, such as steroid-resistant FSGS, or patients having a genetic mutation associated with FSGS, there is a high unmet medical need for new treatments that can reduce proteinuria or induce proteinuria remission and slow down or stop the progression of renal damage and the progression to ESRD in this population.

### BRIEF SUMMARY OF THE INVENTION

Certain embodiments are directed to a method of treating primary focal segmental glomerulosclerosis (primary FSGS) in a patient having an APOL1 variant, comprising administering a TGFβ antagonist, or a pharmaceutical formulation comprising a therapeutically effective amount of a TGFβ antagonist, to the patient.

Certain embodiments are directed to a method of treating primary FSGS in a patient having an APOL1 variant, comprising intravenously administering a TGFβ antagonist, or a pharmaceutical formulation comprising a therapeutically effective amount of a TGFβ antagonist, to a patient.

Certain embodiments are directed to a method of treating primary FSGS in a patient having an APOL1 variant, comprising intravenously administering a TGFβ antagonist, or a pharmaceutical formulation comprising a therapeutically effective amount of a TGFβ antagonist, to a patient, wherein the method stabilizes the patient's glomerular filtration rate.

Certain embodiments are directed to a method of treating primary FSGS in a patient having an APOL1 variant, comprising administering fresolimumab, or a pharmaceutical formulation comprising a therapeutically effective amount of fresolimumab, to the patient.

Certain embodiments are directed to a method of treating primary FSGS in a patient having an APOL1 variant, comprising intravenously administering fresolimumab, or a pharmaceutical formulation comprising a therapeutically effective amount of fresolimumab, to a patient.

Certain embodiments are directed to a method of treating primary FSGS in a patient having an APOL1 variant, comprising intravenously administering fresolimumab, or a pharmaceutical formulation comprising a therapeutically effective amount of fresolimumab, to a patient, wherein the method further comprises stabilizing the patient's glomerular filtration rate.

Certain embodiments are directed to a method of treating primary FSGS in a patient having an APOL1 variant, comprising administering 1-4 mg/kg of fresolimumab, or a pharmaceutical formulation comprising 1-4 mg/kg of fresolimumab, to the patient, on a monthly basis, every 28 days, every 21 days, or every 14 days.

Certain embodiments are directed to a method of treating primary FSGS in a patient having an APOL1 variant, comprising intravenously administering in a single infusion or in a series of infusions over a period of time during a treatment session 1-4 mg/kg of fresolimumab, or a pharmaceutical formulation comprising 1-4 mg/kg of fresolimumab, to the patient, on a monthly basis, every 28 days, every 21 days, or every 14 days.

Certain embodiments are directed to a method of treating primary FSGS in a patient having an APOL1 variant, comprising intravenously administering 1-4 mg/kg of fresolimumab, or a pharmaceutical formulation comprising 1-4 mg/kg of fresolimumab, to the patient, on a monthly basis, every 28 days, every 21 days, or every 14 days, over a 1-50 treatment sessions, such as 1-10 treatment sessions, or 1-4 treatment sessions.

Certain embodiments are directed to a method of treating primary FSGS in a patient having an APOL1 variant, comprising intravenously administering 1-4 mg/kg of fresolimumab, or a pharmaceutical formulation comprising 1-4 mg/kg of fresolimumab, to the patient, on a monthly basis, every 28 days, every 21 days, or every 14 days, wherein the treated patient has a 50% or greater decline in Up/C ratio (mg protein/mg creatinine) from baseline.

Certain embodiments are directed to a method of treating primary FSGS in a patient having an APOL1 variant, comprising intravenously administering 1-4 mg/kg of fresolimumab, or a pharmaceutical formulation comprising 1-4 mg/kg of fresolimumab, to the patient, on a monthly basis, every 28 days, every 21 days, or every 14 days, wherein the treated patient has a 50% or greater decline in Up/C ratio from baseline to a level in the range of from ≥0.3 to ≤3.0 mg protein/mg creatinine.

Certain embodiments are directed to a method of treating primary FSGS in a patient having an APOL1 variant, comprising intravenously administering 1-4 mg/kg of fresolimumab, or a pharmaceutical formulation comprising 1-4 mg/kg of fresolimumab, to the patient, on a monthly basis, every 28 days, every 21 days, or every 14 days, wherein the treated patient has a 50% or greater decline in Up/C ratio from baseline to a level of <0.3 mg protein/mg creatinine.

Certain embodiments are directed to a method of treating primary FSGS in a patient having an APOL1 variant, comprising intravenously administering in a single infusion or in a series of infusions over a period of time during a treatment session 1-4 mg/kg of fresolimumab, or a pharmaceutical formulation comprising 1-4 mg/kg of fresolimumab, to the patient, on a monthly basis, every 28 days, every 21 days, or every 14 days, wherein the APOL1 variant comprises an APOL1 G1 haplotype, such as G1/− or G1/G1, an APOL-1 G2 haplotype, such as G2/− or G2/G2, or a diplotype, such as G1/G2.

Certain embodiments are directed to a method of treating primary FSGS in a patient having an APOL1 variant, comprising intravenously administering in a single infusion or in a series of infusions over a period of time during a treatment session 1-4 mg/kg of fresolimumab, or a pharmaceutical formulation comprising 1-4 mg/kg of fresolimumab, to the patient, on a monthly basis, every 28 days, every 21 days, or every 14 days, wherein the APOL1 variant is homozygous positive for APOL1 G1 haplotype (i.e., G1/G1) or is a heterozygous carrier for APOL1 G1 haplotype (e.g., G1/− or diplotype G1/G2).

Certain embodiments are directed to a method of treating primary FSGS in a patient having an APOL1 variant, comprising intravenously administering in a single infusion or in a series of infusions over a period of time during a treatment session 1-4 mg/kg of fresolimumab, or a pharmaceutical formulation comprising 1-4 mg/kg of fresolimumab, to the patient, on a monthly basis, every 28 days, every 21 days, or every 14 days, wherein the APOL1 variant is homozygous positive for APOL1 G1 haplotype (i.e., G1/G1) or is a heterozygous carrier for APOL1 G1 haplotype (e.g., G1/− or diplotype G1/G2), and wherein the method further comprises genotyping the patient by identifying the presence of the APOL1 G1 haplotype (e.g., G1/G1, G1/−, or G1/G2) in a blood sample isolated from said patient, wherein the genotyping is conducted prior to, during, or after, the treatment.

Certain embodiments are directed to a method of treating primary FSGS in a patient having an APOL1 variant, comprising administering 1-4 mg/kg of fresolimumab, or a pharmaceutical formulation comprising 1-4 mg/kg of fresolimumab, to the patient, on a monthly basis, every 28 days, every 21 days, or every 14 days, wherein the primary FSGS is steroid-resistant primary FSGS.

Certain embodiments are directed to a method of treating primary FSGS in a patient having an APOL1 variant, comprising administering 1-4 mg/kg of fresolimumab, or a pharmaceutical formulation comprising 1-4 mg/kg of fresolimumab, to the patient, wherein the primary FSGS is steroid-resistant primary FSGS, and wherein the APOL1 variant is an APOL1 risk variant comprising G1/G1, G1/−, G1/G2, G2/−, or G2/G2.

Certain embodiments are directed to a method of treating primary FSGS in a patient having an APOL1 variant, comprising administering 1-4 mg/kg of fresolimumab, or a pharmaceutical formulation comprising 1-4 mg/kg of fresolimumab, to the patient, wherein the primary FSGS is steroid-resistant primary FSGS, and wherein the treated patient is of African descent or of Hispanic descent.

### BRIEF DESCRIPTION OF THE FIGURES

FIG. 1. Percent change in mean eGFR (mL/min per 1.73 m²) over time by treatment assignment.

### DETAILED DESCRIPTION OF THE INVENTION

Transforming growth factor beta (TGFβ) is a is a pleiotropic cytokine that belongs to a superfamily of ligands, including bone morphogenetic proteins and activins, which plays an important physiological role in the regulation of cell proliferation and differentiation, extracellular matrix (ECM) production, angiogenesis, wound healing, immune regulation, and embryonic development. As TGFβ plays an important role in renal fibrosis and preclinical models of FSGS have demonstrated prevention of proteinuria and glomerular pathology as well as reduction in pre-existing proteinuria with inhibition of TGFβ, there is reason to believe that fresolimumab may be an effective therapy in patients with steroid-resistant primary FSGS.

In humans, TGFβ exists in 3 isoforms: TGFβ1, β2, and β3. Each isoform is encoded by distinct, highly conserved genes and is a 25 kilodalton homodimeric, disulphide-bonded protein. TGFβ is secreted by cells in a biologically inactive “latent form” by virtue of its association with latency-associated proteins. Much of the TGFβ/latency-associated protein “pro-drug” is stored in the extracellular matrix (ECM); other notable sites include platelet granules and the surface of T-regulatory cells. The mechanism of release of active TGFβ can occur either under acidic conditions (such as within a tumor) or through the action of proteases such as thrombospondin-1, plasmin, or prostate-specific antigen (Dallas S L, et al., “Preferential production of latent transforming growth factor β-2 by primary prostatic epithelial cells and its activation by prostate-specific antigen,” J Cell Physiol., 2005; 202:361-70).

Active TGFβ primarily binds to cells via 2 major receptors, TGFβRII, and TGFβRIII, which may lead to the activation of the Smad pathway, the induction of gene transcription and effects on cell differentiation and growth. Although TGFβ/TGFβR/Smad is the major signaling pathway, TGFβ also binds to other receptors such as endoglin, and other signaling pathways such as ERK, JNK, MAPK, PI3K, Rho-kinase, Akt, and GTPases may also be involved. Downstream effectors of these pathways lead to the physiological and pathological TGF-mediated processes described below (Blobe G C., et al., “Role of transforming growth factor (3 in human disease,” N Engl J Med., 2000; 342:1350-8).

Under normal conditions, members of the TGFβ family maintain homeostasis in many organ systems. In normal and non-cancerous cells, TGFβ limits the growth of epithelial, endothelial, neuronal, and hematopoietic cell lineages through anti-proliferative and apoptotic responses. In addition, TGFβ exerts potent effects that influence immune function, cell proliferation/functional differentiation, cell adhesion, extracellular matrix (ECM) production, cell motility, angiogenesis, and cytokine production (Blobe G C., N Engl J Med., 2000).

Although important physiologically, overexpression of TGFβ may be pathological and contribute to fibrosis, such as renal fibrosis. For example, sustained overactivity of TGFβ may be pathologic and lead to excessive accumulation of ECM, which is the characteristic feature of fibrotic diseases. TGFβ has also been implicated as an important factor in the growth, progression, and metastatic potential of advanced cancers, as well as an important modulator of immune function. The suppression, neutralization, or blockage of TGFβ may ameliorate disease progression in pathological states characterized by excess fibrosis and abnormal cell proliferation, for example FSGS, such as primary FSGS, as well as oncology. In particular, neutralizing or blocking TGFβ with a TGFβ antagonist, such as an antibody directed against TGFβ, may modify the pathologic processes characterized by inappropriate fibrosis, for example FSGS, such as primary FSGS, and may provide a new therapeutic opportunity in treating renal fibrosis.

Preclinical data from several animal models demonstrate that neutralization of TGFβ can inhibit the development of tissue fibrosis. In the rat unilateral ureteral obstruction (UUO) model of renal fibrosis, administration of 1D11, a mouse analogue of the human pan-specific antibody (initially designated as GC-1008) to the 3 major isoforms of transforming growth factor beta (TGFβ; β1, 2, and 3), resulted in significant reductions in collagen deposition and TGFβ expression (Miyajima A, et al., “Antibody to transforming growth factor-β ameliorates tubular apoptosis in unilateral ureteral obstruction,” Kidney Int., 2000; 58:2301-13). In a uninephrectomy rat model of puromycin aminonucleoside (PAN)-induced FSGS, both 1D11 and GC-1008 were effective in preventing the development of proteinuria and glomerular pathology; also 1D11 was effective in reducing pre-existing proteinuria. These results provide evidence that TGFβ antagonism may be effective in preventing and reducing pre-existing proteinuria in an animal model of FSGS.

Fresolimumab (GC-1008) (herein after referred to as “fresolimumab”, and as disclosed in U.S. Pat. No. 7,723,486, U.S. Pat. No. 8,383,780, and U.S. Pat. No. 8,591,901, each of which are herein incorporated by reference in their entirety) is an engineered immunoglobulin subclass gamma 4 (IgG4) human monoclonal antibody directed against the 3 major isoforms of transforming growth factor beta (TGFβ; β1, 2, and 3). Administration of fresolimumab leads to TGFβ neutralization and suppression of TGFβ activity. TGFβ suppression may ameliorate disease progression in pathological states characterized by excess fibrosis and abnormal cell proliferation, for example FSGS, such as primary FSGS.

Certain haplotypes and diplotypes of the APOL1 gene, referred to herein as APOL1 variants or APOL1 risk variants, have been associated with an increased incidence or an increased risk in developing nephropathy, such as non-diabetic nephropathy, for example, primary FSGS, wherein the APOL1 variants (or APOL1 risk variants) include an APOL1 G1 haplotype (e.g., G1/G1 and G1/−), an APOL1 G2 haplotype (e.g., G2/G2 and G2/−), or an APOL1 diplotype (i.e., G1/G2), relative to lacking both G1 and G2 haplotypes (i.e., −/−, or sometimes referred to as “wild type”; which has not been associated with an increased incidence or an increased risk in developing nephropathy, non-diabetic nephropathy, or primary FSGS). (Freedman, Barry I., et al. “Gene-Gene and Gene Environment Interactions in Apolipoprotein L1 Gene-Associated Nephropathy,” Clin. J. Am. Soc. Nephrol., 2014, doi: 10.2215/CJN.01330214).

The APOL1 G1 haplotype, as described in Table 1, is understood to refer to the presence of two specific genetic variants (i.e., coding variants) in the APOL1 gene. People identified as having the APOL1 G1 haplotype, for example people of African descent (or race or ethnicity), have been strongly associated with an increased risk of FSGS disease, such as primary FSGS. The presence of a “G” nucleotide at each position in homozygosity may identify an individual, for example an individual of African descent, as “homozygous positive for the APOL1 G1 haplotype” (i.e., G1/G1), and the individual may have an increased risk of developing primary FSGS. If either or both of the variant positions is/are found in heterozygosity (i.e. A/G and G/G; or G/G and T/G) an individual, for example an individual of African descent, may be identified as a “heterozygous carrier for the APOL1 G1 haplotype”(e.g., G1/−, or diplotype G1/G2) or (A/G and T/G) the individual may be identified as a “potential heterozygous carrier for the APOL1 G1 haplotype” (e.g., G1/−, or diplotype G1/G2), and the individual has a potential risk of developing primary FSGS. Although there is known near-perfect linkage disequilibrium among these two coding variants, the possibility does exist that the variants are not in cis to one another. The absence of an “G” nucleotide at each position in homozygosity may identify an individual, for example an individual of African descent, as “homozygous negative for the APOL1 G1 haplotype” (i.e., −/−), and the individual may be considered to not have an increased risk of developing primary FSGS.

TABLE 1
Coding Variants of APOL1 G1 Haplotype

| | Gene/SNP ID | | |
|---|---|---|---|
| | APOL1/ | APOL1/ | |
| | rs73885319 | rs60910145 | Significance |
| Coding Variant | S342G | I348M | |
| Nucleotide | A > G | T > G | |
| Variation | | | |
| Genotype Result | G/G | T/G | heterozygous carrier for |
| | | | APOL1 G1 haplotype |
| | A/G | G/G | heterozygous carrier for |
| | | | APOL1 G1 haplotype |
| | A/G | T/G | potential heterozygous |
| | | | carrier for APOL1 |
| | | | G1 haplotype |
| | G/G | G/G | homozygous positive for |
| | | | APOL1 G1 haplotype |
| | A/A | T/T | homozygous negative for |
| | | | APOL1 G1 haplotype |

Table Note:
The term “SNP” is understood to refer to herein as a “single nucleotide polymorphism” and defines a specific allele of a given gene.

The APOL1 G2 haplotype, as described in Table 2, is understood to refer to the presence of a specific genetic variant (i.e., coding variant) in the APOL1 gene. People identified as having the APOL1 G2 haplotype, for example people of African descent (or race or ethnicity), have been strongly associated with an increased risk of FSGS disease, such as primary FSGS. The deletion of the 6 bases at the variant position in homozygosity may identify an individual, for example an individual of African descent, as “homozygous positive for the APOL1 G2 haplotype” (i.e., G2/G2), and the individual may have an increased risk of developing primary FSGS. If the variant position is found in heterozygosity the individual may be identified as a “potential heterozygous carrier for the APOL1 G2 haplotype” (e.g., G2/−, or diplotype G1/G2), and has a potential risk of developing primary FSGS. The presence of the 6 bases at the variant position in homozygosity may identify an individual, for example an individual of African descent, as “homozygous negative for the APOL1 G2 haplotype” (i.e., −/−), and not considered to have an increased risk of developing primary FSGS.

TABLE 2
Coding Variants of APOL1 G2 Haplotype

| | Gene/SNP ID | |
|---|---|---|
| | APOL1/rs71785313 | Significance |
| Coding Variant | p.NYK388K | |
| Nucleotide | c.1164_1169delTTATAA | |
| Variation | | |
| Genotype Result | del6/— | heterozygous carrier for |
| | | APOL1 G2 haplotype |
| | del6/del6 | homozygous positive for |
| | | APOL1 G2 haplotype |
| | —/— | homozygous negative for |
| | | APOL1 G2 haplotype |

Table Note:
The term “del6”, as used herein, is understood to refer to a deletion of 6 bases.

There are also SNP variants of APOL1 that are not associated with a haplotype as shown in Table 3). For example, a patient identifed as a heterozygous carrier of the SNP variant rs2239785 of the APOL1 gene is considered to have a potential increase in risk of having or developing FSGS disease, while a patient identifed as a homozygous carrier of the SNP variant rs2239785 of the APOL1 gene is considered to have an increased risk of having or developing FSGS disease.

TABLE 3
SNP Variants Not Associated with an APOL1 Haplotype

| | Nucleotide | | Homozygous | Heterozygous | Homozygous |
|---|---|---|---|---|---|
| Gene/SNP ID | Variation | Coding Variant | non-carrier | carrier | carrier |
| APOL1/rs2239785 | G > A | p.E150K | G/G | A/G | A/A |

In certain embodiments, the method of treating comprises administering a TGFβ antagonist, such as fresolimumab, to a patient that has been diagnosed with nondiabetic nephropathy, nephrotic syndrome, nephrotic proteinuria, nephrotic-range proteinuria, FSGS, or primary FSGS, for example, the patient has been diagnosed as having primary FSGS.

In certain embodiments, the method of treating primary FSGS in a patient, for example a patient of African descent (or race or ethnicity), such as from the African American population, may comprise administering a TGFβ antagonist, such as fresolimumab, to the patient, wherein the patient prior to treatment has an eGFR of 30 mL/min/1.73m² or more, a Up/C ratio of 3 mg protein/mg creatinine or more in at least one urinary sample collected, a Up/C of 2 mg protein/mg creatinine or more in at least two urinary samples collected, or combinations thereof.

In certain embodiments, the present invention related to a method of improving one or more symptoms associated with primary FSGS, comprising administering a TGFβ antagonist, such as a pan-specific TGFβ antagonist, for example, fresolimumab, to a patient, for example a patient of African descent (or race or ethnicity), such as from the African American population, wherein the improvement of the one or more symptoms comprises decreasing proteinuria levels, decreasing Up/C ratio, stabilizing eGFR, reducing the severity of hypoalbuminemia or hyperlipidemia, mitigating edema symptoms, or combinations thereof.

In certain embodiments, the present invention relates to a method of treating primary FSGS in a patient, for example a patient of African descent (or race or ethnicity), such as from the African American population, comprising administering a TGFβ antagonist, such as fresolimumab, to the patient, wherein the patient has steroid-resistant primary FSGS (patient not responsive to steroid treatment) prior to treatment or is intolerant to steroid therapy prior to treatment, for example, the steroid-resistant primary FSGS patient is intolerant to high-dose steroid therapy for at least 2 weeks, such as 3 weeks, 4 weeks, or 5 weeks, prior to treatment. In certain embodiments, the patient to be treated with a TGFβ antagonist has been treated with an ACEi, an ARB, or both, at a stable dose for at least 2 weeks, such as 3 weeks, 4 weeks, or 5 weeks, prior to treatment with the TGFβ antagonist. In certain embodiments, the patient to be treated with a TGFβ antagonist has been treated with an immunosuppressive therapy, prior to, during, or after, treatment with the TGFβ antagonist. In certain embodiments, the patient to be treated with a TGFβ antagonist has been treated with another therapy (such as calcineurin therapy, ciclosporin, tacrolimus, Cyclosporine A, mycophenolate mofetil, rituximab, rosiglitazone, sirolimus (rapamycin), or cyclophosphamide, or combinations thereof), prior to, during, or after, treatment with the TGFβ antagonist. In certain embodiments, the patient to be treated with a TGFβ antagonist has been treated with another therapy (such as calcineurin therapy, ciclosporin, tacrolimus, Cyclosporine A, mycophenolate mofetil, rituximab, rosiglitazone, sirolimus (rapamycin), or cyclophosphamide, or combinations thereof), after treatment with the TGFβ antagonist. In certain embodiments, the patient to be treated with a TGFβ antagonist avoids being treated with another therapy (such as calcineurin therapy, ciclosporin, tacrolimus, Cyclosporine A, mycophenolate mofetil, rituximab, rosiglitazone, sirolimus (rapamycin), or cyclophosphamide, or combinations thereof), prior to or during treatment with the TGFβ antagonist.

In certain embodiments, the method of treating primary FSGS in a patient, for example a patient of African descent (or race or ethnicity), such as from the African American population, or a patient that is of European American descent (or race or ethnicity), such as Caucasian, may comprise administering a TGFβ antagonist, such as fresolimumab, to the patient, wherein the patient has a genetic variant of APOL1 or a genetic variant that increases the patient's risk of developing primary FSGS. In certain embodiments, the genetic variant of APOL1 may be homozygous positive for the APOL1 G1 haplotype (i.e., G1/G1), heterozygous carrier for the APOL1 G1 haplotype (e.g., G1/− or diplotype G1/G2), or homozygous negative for the APOL1 G1 haplotype (i.e., −/−). In certain embodiments, the genetic variant of APOL1 may be homozygous positive for the APOL1 G2 haplotype (i.e., G2/G2), potential heterozygous carrier for the APOL1 G2 haplotype (e.g., G2/− or diplotype G1/G2), or homozygous negative for the APOL1 G2 haplotype (i.e., −/−).

In certain embodiments, the method of treating primary FSGS in a patient, for example a patient of African descent (or race or ethnicity), such as from the African American population, may comprise administering a TGFβ antagonist, such as fresolimumab, to the patient, wherein the patient has a genetic variant of APOL1 that increases the patient's risk of developing primary FSGS. For example, the APOL1 genetic variant that increases the patient's risk of developing primary FSGS may be an APOL1 G1 haplotype (e.g., G1/G1 or G1/−), APOL1 G2 haplotype (e.g., G2/G2 or G2/−), or both haplotypes (i.e., diplotype (G1/G2). In certain embodiments, the APOL1 genetic variant that increases the patient's risk of developing primary FSGS may be homozygous positive for the APOL1 G1 haplotype (G1/G1) or heterozygous carrier for the APOL1 G1 haplotype (G1/− or diplotype G1/G2), for example, the APOL1 genetic variant may be homozygous positive for the APOL1 G1 haplotype (G1/G1). In certain embodiments, the method of treating primary FSGS in a patient, for example a patient of African descent, such as from the African American population, may comprise administering a TGFβ antagonist, such as fresolimumab, to the patient, wherein the patient has an APOL1 G1 haplotype variant comprising SNP ID rs73885319 or rs60910145. In certain embodiments, the APOL1 genetic variant that increases the patient's risk of developing primary FSGS may be homozygous positive for the APOL1 G2 haplotype (G2/G2) or potential heterozygous carrier for the APOL1 G2 haplotype (G2/− or diplotype G1/G2), for example, the APOL1 genetic variant may be homozygous positive for the APOL1 G2 haplotype (G2/G2). In certain embodiments, the method of treating primary FSGS in a patient, for example a patient of African descent (or race or ethnicity), such as from the African American population, may comprise administering a TGFβ antagonist, such as fresolimumab, to the patient, wherein the patient has an APOL1 G2 haplotype variant is SNP ID rs71785313. In certain embodiments, the method of treating primary FSGS in a patient, for example a patient of African descent, such as from the African American population, may comprise administering a TGFβ antagonist, such as fresolimumab, to the patient, wherein the patient has a genetic variant not associated with an APOL1 haplotype for example an APOL1 genetic variant having a SNP ID rs2239785. In certain embodiments, the patient has a genetic variant having a SNP ID selected from the group consisting of: rs73885319, rs60910145, rs71785313, or rs2239785.

In certain embodiments, the patient may be selected on the basis of having at least one indicative SNP of APOL1, for example, APOL1 SNP variants rs2239785, rs73885319, rs60910145, or rs71785313. For example, in certain embodiments, the patient may be selected on the basis of having at least one indicative APOL1 SNP that defines the APOL1 G1 haplotype, such as APOL1 SNP variant rs73885319, or APOL1 SNP variant rs60910145. In certain embodiments, the patient may be selected on the basis of having at least one indicative APOL1 SNP that defines the APOL1 G2 haplotype, such as APOL1 SNP variant rs71785313. In certain embodiments, the patient may be selected on the basis of having at least one indicative APOL1 SNP that can be assessed by allelic discrimination, such as APOL1 SNP variant rs2239785.

In certain embodiments, the method of treating primary FSGS in a patient, for example a patient of African descent (or race or ethnicity), such as from the African American population, may comprise administering a TGFβ antagonist, such as fresolimumab, to the patient, wherein the method further comprises genotyping the patient prior to or during the treatment. For example, the method may further comprises genotyping the patient, comprising: obtaining a biological sample from the patient, wherein said sample is selected from the group consisting of blood, blood-derived product (such as buffy coat, serum, and plasma), lymph, urine, tear, saliva, cerebrospinal fluid, buccal swabs, sputum, hair roots, leukocyte sample or tissue samples or any combination thereof, and identifying the presence or absence of a genetic variant associated with primary FSGS, such as a genetic variant that increases the risk of having or developing primary FSGS. In certain embodiments, the identifying the presence or absence of a genetic variant associated with primary FSGS may include contacting the biological sample with a reagent capable of detecting the genetic variant associated with primary FSGS, by specific direct nucleotide sequencing, by a specific allelic discrimination SNP denotyping assay, or a combination thereof. In certain embodiments, the method further comprises genotyping the patient prior to or during the treatment, comprising obtaining a biological sample from the patient, and identifying the presence or absence of a genetic variant associated with primary FSGS, such as the presence or absence of an APOL1 genetic variant, for example, identifying the presence or absence of an APOL1 G1 haplotype or an APOL1 G2 haplotype, from the biological sample obtained from said patient. For example, the method may further comprise genotyping the patient by identifying the presence of APOL1 G1 homozygotes (G1/G1), the presence of APol 1 G1 heterozygotes (e.g., G1/− or diplotype G1/G2), or the presence of APOL1 G2 heterozygotes (e.g., G2/− or diplotype G1/G2), in a biological sample obtained from said patient, such as a blood sample isolated from said patient. In certain embodiments, the genotyping is conducted prior to the treatment. In certain embodiments, the genotyping is conducted during the treatment.

In certain embodiments, the method of treating may include identification of indicative SNPs of the APOL1 gene, comprising: a) selecting a group of patients having proteinuria levels of ≥3.0 mg protein/mg creatinine; b) obtaining the genotype of said patients at the genetic locus of the APOL1 gene; c) administering to said patients a therapeutic effective amount of an TGFβ antagonist, such as a pan-specific TGFβ antagonist, for example, fresolimumab; d) measuring the levels of proteinuria, eGFR, or both, of the treated patient; e) subdividing the patients of step d) in responder and non-responder subgroups by identifying those patients showing statistically relevant improved levels of proteinuria, eGFR, or both; and f) analyzing the DNA sequence of the genetic loci of the responder and non-responder subpopulations identified in step e) and selecting or interrogating SNPs only present at the genetic loci of the APOL1 geneof the responders; and g) identifying heterozygous or homozygous indicative SNP variants by correlating the existence of the selected or interrogated SNPs with the results of the levels of the proteinuria, eGFR, or both, of step d).

In certain embodiments, the method of treating may include genotyping the patient before, during, or after the treatment period. Methods for conducting genotyping (such as genotyping of SNPs) may include, but are not limited to, DNA sequencing, hybridisation techniques, PCR based assays, fluorescent dye and quenching agent-based PCR assay (Taqman PCR detection system), RFLP-based techniques, single strand conformational polymorphism (SSCP), denaturating gradient gel electrophoresis (DGGE), temperature gradient gel electrophoresis (TGGE), chemical mismatch cleavage (CMC), heteroduplex analysis based system, techniques based on mass spectroscopy, invasive cleavage assay, polymorphism ratio sequencing (PRS), microarrays, a rolling circle extension assay, HPLC-based techniques, DHPLC-based techniques, oligonucleotide extension assays (OLA), extension based assays (ARMS, (Amplification Refractory Mutation System), ALEX (Amplification Refractory Mutation Linear Extension), SBCE (Single base chain extension), a molecular beacon assay, invader (Third wave technologies), a ligase chain reaction assay, 5′-nuclease assay-based techniques, hybridization capillary array electrophoresis (CAE), pyrosequencing, protein truncation assay (PTT), immunoassays, haplotype analysis, and solid phase hydridization (dot blot, reverse dot blot, chips).

In certain embodiments, the method of treating may include administering the TGFβ antagonist, such as a pan-specific TGFβ antagonist, for example, fresolimumab, as an intravenous infusion. For example, the intravenous infusion may be conducted as a single infusion or as series of infusions over a period of time during a treatment session. In certain embodiments, the method of treating may include a single infusion or as series of infusions over a period of time during a treatment session of 50-200 mL, for example, 50-100 mL, 100-150 mL, 75-125 mL, or 150-200 mL, of a pharmaceutical formulation comprising fresolimumab, for example, 1-4 mg/kg of fresolimumab based on the total body weight of the patient, reconstituted in strerile water for injection (sWFI). In certain embodiments, the method of treating may include a single infusion of 100 mL of a pharmaceutical formulation comprising 1-4 mg/kg of fresolimumab based on the total body weight of the patient reconstituted in sWFI. In certain embodiments, the intravenous infusion may take approximately 10 minutes to 1 hour, for example, approximately 20 minutes, approximately 30 minutes, approximately 45 minutes, or approximately 1 hour. For example, the intravenous infusion may be conducted as a single infusion or as series of infusions over a period of time during a treatment session, wherein each infusion of the series of infusions during a treatment session, may take approximately 10 minutes to 1 hour, for example, approximately 20 minutes, approximately 30 minutes, approximately 45 minutes, or approximately 1 hour.

In certain embodiments, the method of treating may include measuring the total body weight of the patient at one or more treatment sessions, such as at the first treatment session, or at each treatment session, to determine the amount of lyophilized fresolimumab reconstituted in sWFI to achieve a 1-4 mg/kg dosing of the fresolimumab based on the total body weight of the patient.

In certain embodiments, the method of treating may include administering the TGFβ antagonist, such as a pan-specific TGFβ antagonist, for example, fresolimumab, as an intravenous infusion on a monthly, biweekly, or periodic basis. For example, in certain embodiments, fresolimumab may be intravenously administered monthly to the patient, for example, intravenously administered every 28 days, every 21 days, or every 14 days, to the patient, at a dose of 1-4 mg/kg based on the total body weight of the patient.

In certain embodiments, the method of treating may include administering a therapeutically effective amount of fresolimumab as an intravenous infusion on a monthly, biweekly, or periodic basis, such as every 28 days, every 21 days, or every 14 days. In certain embodiments, the therapeutically effective amount of the fresolimumab intravenously administered on a monthly, biweekly, or periodic basis, such as every 28 days, every 21 days, or every 14 days, is a dose of 1-4 mg/kg of the fresolimumab based on the total body weight of the patient, for example, the therapeutically effective amount is a dose of 1 mg/kg of the fresolimumab, such as 2 mg/kg, 3 mg/kg, or 4 mg/kg of the fresolimumab, based on the total body weight of the patient. In certain embodiments, the therapeutically effective amount of the fresolimumab is intravenously administered as a pharmaceutical formulation.

In certain embodiments, a pharmaceutical formulation comprising fresolimumab at 1-4 mg/kg total body weight of the patient may be intravenously administered monthly to the patient as a single infusion or as a series of infusions over a period of time during a treatment session, for example, intravenously administered every 28 days, every 21 days, or every 14 days, to the patient as a single infusion or as a series of infusions over a period of time during a treatment session. In certain embodiments, the method of treating may include intravenously administering the TGFβ antagonist, such as a pan-specific TGFβ antagonist, for example, fresolimumab, at a single treatment session or over a series of treatment sessions over the course of months or years. In certain embodiments, the method of treating may include intravenously administering fresolimumab to the patient during a treatment period, which is subsequently followed by a follow-up period, during which no fresolimumab is administered to the patient and the patient is monitored for progress in mitigating symptoms associated with the disease, such as decreasing proteinuria levels, stabilization of eGFR, or reduction of edema symptoms, or combinations thereof. For example, in certain embodiments, the method of treating may include intravenously administering fresolimumab to the patient during a treatment period comprising a series of treatment sessions over the course of 1 month to 5 years on a monthly basis, every 28 days basis, every 21 days basis, or every 14 days basis, for example, over a series of treatment sessions over the course of 1 month to 4 years, 1 month to 3 years, 1 month to 2 years, 1 month to 1 year, 1 month to 12 months, 1 month to 11 months, 1 month to 10 months, 1 month to 9 months, 1 month to 8 months, 1 month to 7 months, 1 month to 6 months, 1 month to 5 months, 1 month to 4 months, 1 month to 3 months, 1 month to 2 months, 2 month to 3 months, 2 month to 4 months, 2 month to 5 months, 2 month to 6 months, 2 month to 7 months, 2 month to 8 months, 3 month to 4 months, 3 month to 5 months, 3 month to 6 months, 4 month to 7 months, 4 month to 9 months, or 5 month to 10 months, on a monthly basis, every 28 days basis, every 21 days basis, or every 14 days basis. For example, in certain embodiments, the method of treating may include intravenously administering fresolimumab to the patient over a series of treatment sessions over the course of 12 months on a monthly basis, every 28 days basis, every 21 days basis, or every 14 days basis, for example, over a series of treatment sessions over the course of 11 months, 10 months, 9 months, 8 months, 7 months, 6 months, 5 months, 4 months, 3 months, 2 months, or 1 month, on a monthly basis, every 28 days basis, every 21 days basis, or every 14 days basis.

In certain embodiments, the method of treating may include intravenously administering fresolimumab to the patient over a series of treatment sessions on a monthly basis, every 28 days basis, every 21 days basis, or every 14 days basis, followed by a treatment holiday, and then followed by a further series of treatment sessions on a monthly basis, every 28 days basis, every 21 days basis, or every 14 days basis. In certain embodiments, the method of treating may include a repeating alternating sequence of a series of treatement sessions, followed by treatement holidays, over the course of 1-10 years, for example, 1-7 years, such as 1-5 years or 1-3 years. For example, in certain embodiments, the method of treating may include intravenously administering fresolimumab to the patient over a series of treatment sessions over the course of 12 months on a monthly basis, every 28 days basis, every 21 days basis, or every 14 days basis, followed by a treatment holiday of 3 months to 5 years, and then followed by a further series of treatment sessions over the course of 12 months on a monthly basis, every 28 days basis, every 21 days basis, or every 14 days basis. For example, the treatment holiday between a first treatment series and a further treatment series may be 3 months to 5 years, for example, 3 months to 4 years, such as 3 months to 3 years, 3 months to 2 years, 3 months to 1 year, 3 months to 9 months, or 3 months to 6 months.

In certain embodiments, the method of treating may include intravenously administering the TGFβ antagonist, such as a pan-specific TGFβ antagonist, for example, fresolimumab, at a single treatment session or over a series of treatment sessions over the course of 1 month to 5 years on a monthly basis, every 28 days basis, every 21 days basis, or every 14 days basis, for example, over a series of 1-50 treatment sessions, such 1-40 treatment sessions, 1-30 treatment sessions, 1-20 treatment sessions, 1-15 treatment sessions, 1-10 treatment sessions, 1-9 treatment sessions, 1-8 treatment sessions, 1-7 treatment sessions, 1-6 treatment sessions, 1-5 treatment sessions, 1-4 treatment sessions, 1-3 treatment sessions, 1-2 treatment sessions, 2-10 treatment sessions, 2-8 treatment sessions, 2-6 treatment sessions, 2-5 treatment sessions, 2-4 treatment sessions, 3-10 treatment sessions, 3-8 treatment sessions, 3-6 treatment sessions, 3-5 treatment sessions, 3-4 treatment sessions, 4-10 treatment sessions, 4-8 treatment sessions, 4-7 treatment sessions, 4-6 treatment sessions, 4-5 treatment sessions, 5-10 treatment sessions, 7-15 treatment sessions, or 15-20 treatment sessions, over the course of 1 month to 5 years on a monthly basis, every 28 days basis, every 21 days basis, or every 14 days basis. In certain embodiments, the method of treating may include intravenously administering fresolimumab, at a single treatment session or over a series of treatment sessions, such as over a series of 10 treatment sessions, 9 treatment sessions, 8 treatment sessions, 7 treatment sessions, 6 treatment sessions, 5 treatment sessions, 4 treatment sessions, 3 treatment sessions, or 2 treatment sessions, over the course of 1 month to 5 years on a monthly basis, every 28 days basis, every 21 days basis, or every 14 days basis.

In certain embodiments, the method of treating may include intravenously administering fresolimumab at a sufficient dose to achieve a trough serum plasma level of fresolimumab of 100 to 51,500 ng/mL within the range of 21 to 84 days from the time of administration, such as within the range of 21 to 42 days, 28 to 49 days, 35 to 56 days, or 49 to 84 days, from the time of administration, for example, 200 to 51,500 ng/mL, 500 to 51,500 ng/mL, 1,000 to 51,500 ng/mL, 5,000 to 51,500 ng/mL, 10,000 to 51,500 ng/mL, 25,000 to 51,500 ng/mL, 100 to 30,000 ng/mL, 100 to 15,000 ng/mL, 103 to 51,209 ng/mL, or 10,000 to 40,000 ng/mL within the range of 21 to 84 days from the time of administration, such as within the range of 21 to 42 days, 28 to 49 days, 35 to 56 days, or 49 to 84 days, from the time of administration. For example, in certain embodiments, the trough serum plasma level of fresolimumab of a treated patient is 1,000 to 51,500 ng/mL, 1,000 to 45,000 ng/mL, 1,000 to 35,000 ng/mL, 1,000 to 25,000 ng/mL, 1,000 to 15,000 ng/mL, 5,000 to 40,000 ng/mL, 10,000 to 51,500 ng/mL, 15,000 to 51,500 ng/mL, 1,813 to 51,209 ng/mL. or 2,000 to 51,500 ng/mL within the range of 21 to 84 days from the time of administration, such as within the range of 21 to 42 days, 28 to 49 days, 35 to 56 days, or 49 to 84 days, from the time of administration.

In certain embodiments, the method of treating may result in decreasing the level of proteinuria in said treated patient from baseline. In certain embodiments, the method of treating may result in the treated patient having a 40% or greater decline in urinary total protein: creatinine ratio (mg protein/mg creatinine), also referred to as “Up/C ratio”, from baseline, for example, a 45% or greater, such as 50% or greater, 55% or greater, 60% or greater, 65% or greater, or 70% or greater decline in Up/C ratio from baseline after 2 treatment sessions, for example, after 3, 4, 5, 6, 7, 8, 9, or 10 treatment sessions. In certain embodiments, the method of treating may result in the treated patient having a 40% or greater decline in Up/C ratio from baseline after 2 treatment sessions, for example, after 3, 4, 5, 6, 7, 8, 9, or 10 treatment sessions, such as a 50% or greater decline in Up/C ratio from baseline, and the Up/C ratio is to a level in the range of from ≥0.3 to ≤3.0 mg protein/mg creatinine after 2 treatment sessions, for example, after 3, 4, 5, 6, 7, 8, 9, or 10 treatment sessions, for example, the Up/C ratio is to a level in the range of from >0.3 to <1.0 mg protein/mg creatinine, such as ≥0.3 to ≤2.0, ≥0.5 to ≤1.5, >1.0 to ≤2.0, ≥1.5 to ≤2.5, ≥1.0 to ≤3.0, or ≥2.0 to ≤3.0 mg protein/mg creatinine after 2 treatment sessions, for example, after 3, 4, 5, 6, 7, 8, 9, or 10 treatment sessions.

In certain embodiments, the method of treating may result in the treated patient having a 40% or greater decline in Up/C ratio from baseline, such as a 50% or greater decline in Up/C ratio from baseline, and the Up/C ratio is to a level of <0.3 mg protein/mg creatinine. In certain embodiments, the method of treating may result in the treated patient having a 40% or greater decline in Up/C ratio from baseline, such as a 50% or greater decline in Up/C ratio from baseline, to a level in the range of from ≥0.3 to ≤3.0 mg protein/mg creatinine after 2 treatment sessions, for example, after 3, 4, 5, 6, 7, 8, 9, or 10 treatment sessions. In certain embodiments, the method of treating may result in the treated patient having a 40% or greater decline in Up/C ratio from baseline, such as a 50% or greater decline in Up/C ratio from baseline, to a level in the range of from ≥0.3 to ≤3.0 mg protein/mg creatinine during the follow-up period, i.e., occurring after the treatment period. In certain embodiments, the method of treating may result in the treated patient having a 40% or greater decline in Up/C ratio from baseline, such as a 50% or greater decline in Up/C ratio from baseline, to a level of <0.3 mg protein/mg creatinine after 2 treatment sessions, for example, after 3, 4, 5, 6, 7, 8, 9, or 10 treatment sessions. In certain embodiments, the method of treating may result in the treated patient having a 40% or greater decline in Up/C ratio from baseline, such as a 50% or greater decline in Up/C ratio from baseline, to a level of <0.3 mg protein/mg creatinine during the follow-up period, i.e., occurring after the treatment period.

In certain embodiments, the method of treating may result in the treated patient having at least one of the following during the treatment period or the follow-up period: i) at least two 50% or greater declines in Up/C ratio from baseline to a level in the range of from ≥0.3 to ≤3.0 mg protein/mg creatinine; or ii) at least one 50% or greater decline in Up/C ratio from baseline to a level in the range of from ≥0.3 to ≤3.0 mg protein/mg creatinine and the Up/C ratio remains at least 50% or less of baseline level for at least one month. For example, in certain embodiments, the method of treating may result in the treated patient having at least two 50% or greater declines in Up/C ratio from baseline to a level in the range of from ≥0.3 to ≤3.0 mg protein/mg creatinine, for example, the Up/C ratio is to a level in the range of from ≥0.3 to ≤1.0 mg protein/mg creatinine, such as ≥0.3 to ≤2.0, ≥0.5 to ≤1.5, ≥1.0 to ≤2.0, ≥1.5 to ≤2.5, ≥1.0 to ≤3.0, or ≥2.0 to ≤3.0 mg protein/mg creatinine, within 112-224 days from starting the treatment, such as 112-196 days, 112-168 days, or within 112-140 days, from starting the treatment.

For example, in certain embodiments, the method of treating may result in the treated patient having at least one 50% or greater decline in Up/C ratio from baseline to a level in the range of from ≥0.3 to ≤3.0 mg protein/mg creatinine, for example, the Up/C ratio is to a level in the range of from ≥0.3 to ≤1.0 mg protein/mg creatinine, such as ≥0.3 to ≤2.0, ≥0.5 to ≤1.5, ≥1.0 to ≤2.0, ≤1.5 to ≤2.5, ≥1.0 to ≤3.0, or ≥2.0 to ≤3.0 mg protein/mg creatinine, and the Up/C ratio remains at least 50% or less of baseline level for at least 2 week, for example at least 3 weeks, such as for at least one month, at least 5 weeks, at least 6 weeks, at least 8 weeks, or for at least 12 weeks, within 112-224 days from starting the treatment, such as 112-196 days, 112-168 days, or within 112-140 days, from starting the treatment.

In certain embodiments, the method of treating may result in stabilizing the kidney function of the treated patient, such as stabilizing the estimated glumerular filtration rate (referred to as “eGFR”) of the treated patient. In certain embodiments, stabilization of a treated patient's eGFR is achieved when the treated patient's median eGFR declines 15% or less from the baseline levels, for example, when the treated patient's median eGFR declines less than 15%, such as declines less than 12%, declines less than 10%, declines less than 9%, declines less than 8%, declines less than 7%, declines less than 6%, declines less than 5%, declines less than 4%, or declines less than 2% from the baseline levels. In certain embodiments, the median eGFR of the treated patient is stabilized at 15% or less, such as 10% or less, from the baseline level, and the eGFR stabilization occurs within 112-224 days from starting the treatment, such as 112-196 days, 112-168 days, or within 112-140 days, from starting the treatment.

In certain embodiments, the method of treating may result in mitigating or minimizing the severity of one or more symptoms associated with nephrotic syndrome such as primary FSGS, comprising hypoalbuminemia, edema, or hyperlipidemia. For example, the method of treating may result in mitigating or minimizing the severity of one or more symptoms associated with primary FSGS in a patient having nephrotic-range proteinuria (e.g., ≥3.0 g/24 h or ≥3.0 mg protein/mg creatinine) or more severe proteinuria (e.g., ≥10 g/24 h).

In certain embodiments, the treated patient may be a patient of African descent (or race or ethnicity), such as from the African American population, a patient that is of European American descent (or race or ethnicity), such as Caucasian, or a patient of Hispanic descent (or race or ethnicity).

### Pharmaceutical Formulations

In certain embodiments, the method of treating may include administering the TGFβ antagonist, such as a pan-specific TGFβ antagonist, for example, fresolimumab, as a pharmaceutical formulation. In certain embodiments, the pharmaceutical formulation may comprise a lyophilized TGFβ antagonist product, such as a lyophilized TGFβ antagonist product that is reconstituted in sWFI. In certain embodiments, the pharmaceutical formulation may comprise lyophilized fresolimumab reconstituted in sWFI, for example, reconstituted in 3.0-10.0 mL sWFI, such as reconstituted in 4.0-8.0 mL sWFI, reconstituted in 4.0-6.0 mL sWFI, reconstituted in 4.5-5.5 mL sWFI, reconstituted in 4.0 mL sWFI, reconstituted in 5.0-5.5 mL sWFI, reconstituted in 5.0-5.3 mL sWFI, reconstituted in 5.0 mL sWFI, reconstituted in 5.1 mL sWFI, reconstituted in 5.2 mL sWFI, reconstituted in 5.3 mL sWFI, reconstituted in 5.4 mL sWFI, reconstituted in 5.5 mL sWFI, or reconstituted in 6.0 mL sWFI. In certain embodiments, the lyophilized fresolimumab may be stored in a vial, for example, 50 mg/vial, and reconstituted in 3.0-10.0 mL sWFI and, for example, stored in a 50 mg/vial and reconstituted in 5.0-5.3 mL sWFI, such as 5.0 mL sWFI, 5.1 mL sWFI, 5.2 mL sWFI, or 5.3 mL sWFI. In certain embodiments, the lyophilized fresolimumab may be reconstituted in 5.1 mL sWFI at 50 mg/vial.

### EXAMPLES

Test Product refers to fresolimumab, an engineered human IgG4 kappa monoclonal antibody capable of neutralizing all mammalian isoforms of TGFβ (i.e., β1, 2, and 3).

Test Pharmaceutical Formulation refers to the intravenous formulation of lyophilized fresolimumab powder reconstituted with 5.1 mL (50 mg/vial) of sterile water for injection (sWFI), wherein the dose strength of 1 mg/kg or 4 mg/kg of the lyophilized fresolimumab powder is based on the total body weight of the patient at each visit. The composition of the lyophilized fresolimumab powder is listed in Table 4.

TABLE 4
Composition of the Lyophilized Powder

| | Amount (mg) per | |
|---|---|---|
| Component | 50 mg Vial | Function |
| Fresolimumab | 53 | Active Ingredient |
| Mannitol, USP/EP | 159 | Stabilizer |
| Sucrose, NF | 53 | Stabilizer |
| Polysorbate 80, NF | 0.53 | Stabilizer |
| Sodium phosphate, dibasic | 46.6 | Buffer |
| heptahydrate, USP | | |
| Sodium phosphate, monobasic | 12.7 | Buffer |
| monohydrate, USP | | |
| Sodium chloride, USP | 7.7 | Buffer |
| Nitrogen, NF | | Inert Gas |
| Approximate weight of lyophilized | 334 mg | |
| cake | | |

Table Note:
USP: United States Pharmacopeia;
EP: European Pharmacopeia;
NF: National Formulary

Prior to administration, lyophilized fresolimumab was reconstituted with 5.1 mL (50 mg vial) of sWFI to result in a protein concentration of approximately 10 mg/mL in a 50 mM sodium phosphate buffer at pH 7.1, containing 25 mM sodium chloride, 3% mannitol, 1% sucrose, and 0.01% polysorbate 80. Filter needles must not be used to remove reconstituted drug product from the vial. However, due to the nature of proteins and their ability to precipitate, the use of a 0.22 μm low protein binding inline filter was required when administering this product (diluted as a solution for infusion). The composition of the Test Pharmaceutical Formulation prepared from the reconstituted fresolimumab is listed in Table 5.

TABLE 5
Composition of Reconstituted Fresolimumab

| | Concentration |
|---|---|
| Component | (mg/mL) |
| Fresolimumab | 10 |
| Mannitol | 30 |
| Sucrose | 10 |
| Polysorbate 80 | 0.1 |
| Sodium phosphate, dibasic heptahydrate | 8.8 |
| Sodium phosphate, monobasic monohydrate | 2.3 |
| Sodium chloride | 1.5 |

Vials containing fresolimumab must be stored at 2 to 8° C. (35.6 to 46.4° F.) until preparation for infusion. Reconstituted fresolimumab is stable after reconstitution with sWFI, for example, stable for up to 24 hours after reconstitution with sWFI, at either room temperature or under refrigeration (between 2 to 8° C. or 35.6 to 46.4° F.). Although stable for up to 24 hours under these conditions, fresolimumab in sWFI should be used immediately. Reconstituted fresolimumab in sWFI that is further diluted in dextrose 5% in water at a concentration of 0.3 mg/mL to 7 mg/mL is stable for up to 24 hours at room temperature.

Placebo formulation refers to the intravenous formulation of lyophilized placebo product (which visually matches the test product) reconstituted with 5.1 mL (50 mg/vial) of sterile water for injection (sWFI).

Route of administration was conducted as a single 100 mL intravenous infusion administered over approximately 30 minutes.

Dose regimen included four (4) monthly administrations of the test formulation or placebo formulation by infusion on Day 1, Day 28, Day 56, and Day 84, wherein the dose strength was calculated based on the total body weight of the patient at each visit.

### Example 1

A double-blind, parallel-dosing, randomized study of two (2) doses of the Test Pharmaceutical Formulation or placebo in patients with steroid-resistant FSGS, in which the study was divided into 3 periods:

1. Screening (Visit 1, up to 6 weeks);

2. Treatment Period (Day 1/Visit 2, Day 28/Visit 3, Day 56/Visit 4, Day 84/Visit 5, and Day 112/Visit 6); and

3. Follow-up Period (Day 140/Visit 7, Day 168/Visit 8, and Day 252/Visit 9).

On Day 1/Visit 2, Day 28/Visit 3, Day 56/Visit 4, and Day 84/Visit 5 patients received a single intravenous (IV) Test Pharmaceutical Formulation or placebo infusion in the clinic. The infusion duration was approximately 30 minutes following which the patients remained in the clinic for an observation period of at least 30 minutes. Patients were scheduled to return to the clinic on Day 112/Visit 6 through Day 252/Visit 9 for follow-up visits.

Renal assessments (proteinuria, serum creatinine, and eGFR), serum lipids, serum albumin, and weight were collected at each visit. Patient reported outcomes were assessed based on a questionnaire completed at Day 1/Visit 2, Day 56/Visit 4, Day 112/Visit 6/early termination (ET), and Day 252/Visit 9. Safety data, including safety laboratory assessments, physical examinations, and vital signs were collected throughout the study. All adverse events (AEs) and concomitant medications were collected during the Treatment Period. Only drug-related and protocol-related serious adverse events (SAEs) and all medical events of interest (MEOIs) were scheduled to be collected during the Follow-up Period. Only medications to treat FSGS were collected during the Follow-up Period.

Blood samples for the determination of the Test Product serum concentrations were collected at each Treatment Period and Follow-up Period visit.

Potential development of anti-fresolimumab antibodies was also evaluated at Day 1/Visit 2, Day 112/Visit 6/ET, Day 140/Visit 7, Day 168/Visit 8, and Day 252/Visit 9.

A total of 36 patients with treatment-resistant FSGS were randomized to treatment with Test Pharmaceutical Formulation at 1 mg/kg (n=14), Test Pharmaceutical Formulation at 4 mg/kg (n=12), or placebo formulation (n=10). The majority of patients (92%) completed all 4 study infusions and no patients discontinued from the study during the treatment period. One patient in the Test Pharmaceutical Formulation 1 mg/kg arm completed 3 infusions, one patient in the Test Pharmaceutical Formulation 4 mg/kg arm completed 3 infusions, and one patient in the Test Pharmaceutical Formulation 4 mg/kg arm completed 2 infusions. Reasons for not completing all study treatments included Physician Decision (to start another therapy) for the patient in the Test Pharmaceutical Formulation 1 mg/kg arm, and adverse events of mild liver enzyme abnormalities and kidney failure (both assessed as not related to treatment) in the 2 patients in the Test Pharmaceutical Formulation 4 mg/kg arm. All 36 patients completed the Treatment Period (up through Day 112/Visit 6). All patients except for one (assigned to the Test Pharmaceutical Formulation at 4 mg/kg arm and was lost to follow-up prior to conclusion of the study on Day 252) completed study visits during the follow up period. The diagnosis and key criteria for including patients having Steroid-Resistant Primary FSGS with nephrotic proteinuria included the following:
i) The patient was willing and able to provide signed informed consent, and was a man or woman 18 years of age or above at time of giving consent.
ii) The patient's renal biopsy was evaluated and was consistent with the diagnosis of primary FSGS including all histological subtypes.
iii) The patient had a calculated eGFR ≥30 mL/min/1.73 m² at Visit 1.
iv) The patient had a Up/C ratio ≥3 (mg protein/mg creatinine) in at least 1 of the urine samples collected during the Screening Period and a Up/C ratio ≥2 (mg protein/mg creatinine) in 2 samples during the Screening Period.
v) In the opinion of the Investigator, the patient had steroid-resistant FSGS. The patient must have been treated for FSGS with a course of high-dose steroid therapy for a minimum of 4 weeks in duration or have been found to be intolerant to steroids prior to the Screening visit.
vi) The patient had been treated with an ACEi and/or ARB at a stable dose for a minimum of 4 weeks prior to Visit 2 (treatment start) unless intolerant or contraindicated.

Patient Demographics: The median age of the patient population was 41 years (range 19 to 77 years), and was consisted of 53% males and 47% females. The race of the patient population was approximately 83% Caucasian and approximately 17% Black, and the ethnicity consisted of approximately 31% Hispanic or Latino and approximately 69% not Hispanic or Latino. The demographic and clinical characteristics as well as baseline proteinuria and kidney function by eGFR were generally balanced across the 3 treatment arms. There were fewer patients who received past Calcineurin (CNI) Therapy in the Test Pharmaceutical Formulation at 1 mg/kg arm (57.1%) compared to the Test Pharmaceutical Formulation at 4 mg/kg arm (75%) or placebo (90%). There were 3 patients in the Test Pharmaceutical Formulation at 1 mg/kg arm with history of thrombosis (possibly signifying more severe disease associated with nephrotic syndrome) and none in the other two treatment assignments.

The median time from initial kidney biopsy diagnosis of the patient population was 3.0 years (range 0.1 to 16.8 years), and approximately 72% of the patients had previously received calcineurin therapy for their FSGS disease. Most patients had received multiple courses of different types of immunosuppressive medications in addition to high dose corticosteroid therapy and approximately two-thirds of treatments were reported as resulting in “no response” with the remainder being recorded as transient and/or partial responses. FSGS histologic subtypes were reasonably well balanced between the three treatment arms.

Past and current medication use for FSGS and response: All patients had received high dose corticosteroid therapy for ≥4 weeks for FSGS disease. Of the 36 randomized patients, there were a total of 123 corticosteroid treatment courses with 66 (53.7%) reported as “No response”, 36 (29.3%) reported as “Transient Partial Response”, 8 (6.5%) reported as “Sustained Partial Response”, 2 (1.6%) reported as Transient Complete Response, and 11 (8.9%) reported as “Patient Intolerant of Drug”. Previous response types did not vary meaningfully across the 3 treatment arms.

Other non-steroid immunosuppressant medication use consisted of multiple medications (including calcineurin inhibitors ciclosporin and tacrolimus) with some patients having received several different previous types with overall insufficient proteinuria reduction with the majority of treatment courses (72 out of 107 or 67.3%) categorized as “No response” in all enrolled patients. Previous response types to non-steroid immunosuppressant medications did not vary meaningfully across the three treatment arms.

Characterization of the patients included in the study, detailing the demographic and baseline disease characteristics, are presented in Table 6 (results presented as median (min, max) or n (%)).

TABLE 6
Demographic and Baseline Characteristics

| | Test Pharmaceutical | | | | | | | |
|---|---|---|---|---|---|---|---|---|
| | Formulation | | | | | | All | |
| | 1 mg/kg | | 4 mg/kg | | | | randomized | |
| | (N = 14) | | (N = 14) | | Placebo (N = 10) | | patients (N = 36) | |
| Age (years) | 50.0 | (22, 76) | 37.5 | (23, 64) | 42.0 | (19, 75) | 40.5 | (19, 76) |
| Male sex | 7 | (50%) | 6 | (50%) | 6 | (60%) | 19 | (52.8%) |
| Race/ethnicity | | | | | | | | |
| Caucasian | 12 | (85.7%) | 10 | (83.3%) | 8 | (80%) | 30 | (83.3%) |
| Black | 2 | (14.3%) | 2 | (16.7%) | 2 | (20%) | 6 | (16.7%) |
| Ethnicity | | | | | | | | |
| Hispanic or Latino | 6 | (42.9%) | 2 | (16.7%) | 3 | (30%) | 11 | (30.6%) |
| Not Hispanic or Latino | 8 | (57.1%) | 10 | (83.3%) | 7 | (70%) | 25 | (69.4%) |
| Weight (kg) | 78.4 | (55, 128.9) | 75.0 | (55, 114.8) | 88.6 | (58.1, 122.5) | 77.4 | (55, 128.9) |
| Height (cm) | 165 | (152, 185) | 168.5 | (157, 188) | 174 | (157, 187) | 168 | (152, 188) |
| BMI (kg/m2) | 29.0 | (19.5, 50.4) | 25.1 | (21.2, 46.6) | 29.5 | (17.5, 43.7) | 27.4 | (17.5, 50.4) |
| Hypertension | 9 | (64.3%) | 8 | (66.7%) | 7 | (70%) | 24 | (66.7%) |
| History of thrombosis | 3 | (21.4%) | 0 | (0%) | 0 | (0%) | 3 | (8.3%) |
| Family history of renal disease | 5 | (35.7%) | 2 | (16.7%) | 3 | (30%) | 10 | (27.8%) |
| Years since FSGS diagnosis | 2.9 | (0.2, 16.8) | 3.02 | (0.1, 12.2) | 3.38 | (0.1, 13.9) | 3.02 | (0.1, 16.8) |
| Renal function decline since FSGS | 4 | (28.6%) | 5 | (41.7%) | 4 | (40.0%) | 13 | (36.1%) |
| diagnosis (YES) | | | | | | | | |
| FSGS Histologic Subtypes | | | | | | | | |
| NOS | 8 | (57.1%) | 4 | (33.3%) | 6 | (60%) | 18 | (50%) |
| Cellular | 0 | (0%) | 2 | (16.7%) | 0 | (0%) | 2 | (5.6%) |
| Collapsing | 2 | (14.3%) | 3 | (25%) | 10 | (10%) | 6 | (16.7%) |
| Perihilar | 1 | (7.1%) | 0 | (0%) | 1 | (10%) | 2 | (5.6%) |
| Tip | 2 | (14.3%) | 3 | (25%) | 2 | (20%) | 7 | (19.4%) |
| None¹ | 1 | (7.1%) | 0 | (0%) | 0 | (0%) | 1 | (2.8%) |
| Patients with prior Calcineurin (CNI) | 8 | (57.1%) | 9 | (75%) | 9 | (90%) | 26 | (72.2%) |
| Therapy² (reported in eCRF) | | | | | | | | |
| Patients with prior Calcineurin (CNI) | 6 | (42.9%) | 5 | (41.7%) | 4 | (40%) | 15 | (41.7%) |
| Therapy² (used for stratification in | | | | | | | | |
| IVRS) | | | | | | | | |
| Prior non-steroid immunosuppressant | | | | | | | | |
| treatments | | | | | | | | |
| Cyclosporin A | 7 | (50%) | 7 | (58.3%) | 7 | (70%) | 21 | (58.3%) |
| Tacrolimus | 3 | (21.4%) | 6 | (50%) | 4 | (40%) | 13 | (36.1%) |
| Mycophenolate (mofetil or acid) | 4 | (28.6%) | 7 | (58.3%) | 4 | (40%) | 15 | (41.7%) |
| Adalimumab | 0 | (0%) | 1 | (8.3%) | 0 | (0%) | 1 | (2.8%) |
| Azathioprine | 0 | (0%) | 1 | (8.3%) | 0 | (0%) | 1 | (2.8%) |
| Cyclophosphamide | 0 | (0%) | 2 | (16.7%) | 3 | (30%) | 5 | (13.9%) |
| Rituximab | 0 | (0%) | 2 | (16.7%) | 2 | (20%) | 4 | (11.1%) |
| Screening eGFR (ml/min/1.73 m2) | 58 | (32, 103) | 68 | (36, 146) | 60 | (33, 159) | 63 | (32, 159) |
| Baseline (Visit 2) UP/C (mg | 5.92 | (2.6, 17.3) | 6.46 | (1.3, 15.9) | 6.41 | (2.2, 13.7) | 6.19 | (1.3, 17.3) |
| protein/mg Cr) | | | | | | | | |

¹One patient was initially not considered primary FSGS due to some glomerular basement membrane abnormalities seen, but further discussion with clinician and central pathologist concluded that the clinical presentation was consistent with sudden onset nephrotic syndrome (not explained by basement membrane findings) and primary FSGS could not be definitively ruled out and patient was enrolled into the study; variant of FSGS lesions seen on biopsy were noted to be NOS.
²Previous CNI therapy status as recorded in eCRF has been determined to be accurate and correlated with previous CNI medication use listed in Past Medications.

Genotyping Data: APOL1 genotyping was performed in 35 of the 36 randomized patients (genotype of patient 4A was not obtained), using either Genotyping Procedure (A) or Procedure (B), as detailed below.

Genotyping Procedure (A)—Allelic Discrimination:

The determination and evaluation of eight (8) genetic variants of APOL1 associated with FSGS (Bostrom, M. A., et al., “Genetic association and gene-gene interaction analyses in African American dialysis patients with nondiabetic nephropathy,” Am J Kidney Dis., 2012, 59, 210-221.; and Genovese, G., et al., “A risk allele for focal segmental glomerulosclerosis in African Americans is located within a region containing APOL1 and MYH9,” Kidney Int., 2010, 78, 698-704) by specific Allelic Discrimination SNP Genotyping Assays (Applied Biosystems, Inc.) is outlined in Table 7:

TABLE 7

| | | | | Allele Determination** | | |
|---|---|---|---|---|---|---|
| | | | | VIC ® detector/ | | FAM ™ detector/ |
| SNP | rs#/ | | | Homozygous | | Homozygous |
| ID | mutation | Gene | Assay ID* | Allele “1” | Heterozygous | Allele “2” |
| A | rs2239785/ | APOL1 | C_1 5796743_10 | A/A | A/G or G/A | G/G |
| | c.448G > A | | | | | |

Table Note:
Assay ID* - TaqMan ® SNP Genotyping Assay, Applied Biosystems, Inc., Catalog #, each at 40X Medium Scale, Part #4332072, Carlsbad, CA;
**FAM ™ and VIC ® from Applied Biosystems, Inc.

Additional reagents utilized to determine the eight (8) genetic variants detailed in Table 7 includes:

Genomic DNA or plasmid samples known to represent the three potential genotypes for each variant were utilized as PCR controls. A sample that tests appropriately may be used for up to 2 years.

2× TaqMan® Universal PCR Mastermix (Applied Biosystems, Inc, Catalog #4326708 Carlsbad, Calif.).

Nuclease Free-water (Ambion, Catalog# AM9932 or equivalent Carlsbad, Calif.).

40× TaqMan SNP Genotyping Assay Single Use Aliquots (12 μL).

2× TaqMan Universal PCR Mastermix Single Use Aliquots (650 μL).

Equipment utilized: Applied Biosystems 7900 Real-Time PCR system with Applied Biosystems Sequence Detection Software version 2.4 or equivalent (Applied Biosystems).

Procedure: In general, the procedure involves diluting the genomic DNA from the patient to a final concentration of 5 ng/mL, which is then used as a template for 40-cycle quantitative PCR in the presence of a single SNP genotyping assay and 2X TaqMan Universal PCR Mastermix according to the instructions provided by manufacturer and as further detailed below. Determinations of the genotype are generated by a Quantstudio 12K Flex Real-Time PCR instrument running software v1.2.2 (LifeTechnologies). The procedure further involves the following steps (a genotyping run takes approximately 1.5 hours to complete):
1—A whole blood sample is collected (drawn) from a patient.
2—Process the whole blood sample for the isolation of DNA, producing two (2) DNA aliquots if possible.
3—Qualification of the Allelic Discrimination assay(s) lot is verified.
4—Samples and controls is diluted to 5 ng/μL in nuclease-free water prior to analysis.
5—Three characterized DNA samples serve as controls for each of the SNPs of interest, if available. Each is representative of a specific genotype: homozygous Allele “1”, heterozygous, and homozygous Allele “2”. Positive controls are run once for each SNP.
6—For each patient, genotype each of the eight (8) SNPs in duplicate according to the following sampling plan: If two DNA aliquots are available, sample and analyze each aliquot once; if only one DNA aliquot is available, sample and analyze in duplicate.
7—The Mastermix(es) are prepared, and briefly vortexed to mix, in a pre-PCR hood using the following reagent volumes (provided as single reaction volumes): 40× ABI SNP Genotyping Assay (0.63 μL); 2X TaqMan Universal PCR Master Mix (12.50 μL); and Nuclease-free water (9.90 μL); such that the final volume was 23.03 μL.
8—The bottle of2× TaqMan®Universal PCR Master Mix is briefly vortexed gently to mix.
9—The 40× genotyping assay is vortexed for 5-10 seconds and nanofuged for 5-10 seconds to return contents to the bottom of the tube.
10—The reagents are combined in a nuclease-free tube, and vortexed briefly to mix.
11—A Plate Map is prepared and completed, indicating the positions of samples and controls.
12—Aliquot 23 μL of the appropriate mastermix is placed into each experimental well according to a completed Plate Map.
13—Add 2 μL water blank (NTC) to the appropriate wells based on the Plate Map.
14—Plate is sealed and centrifuged at 1,250 ±250g for 15-30 seconds.
15—Sample Addition: using a new pipet tip for each sample, pipet 2.0 μL of DNA (diluted to 5 ng/μL) is added to the appropriate well based on the completed Plate Map. Pipette up and down to mix.
16—Add 2 μL each control (diluted to 5 ng/μL) specific to each SNP assay, to the appropriate wells referenced on the Plate Map. Pipette up and down to mix.
17—The optical plate is sealed using the optical adhesive film, and then centrifuged at 1,250±250 g for 15-30 seconds.
18—Data collection: use compressed air (Whoosh Duster) to ensure that no dust or debris is contaminating the exterior of the plate prior to installing it into the plate adapter in the QuantStudio™ 12K Flex Real-time PCR instrument.
19—A new genotyping “experiment” file (.eds) is created and run using the set up parameters defined as follows (using the instrument's touch panel or from the QuantStudio™ 12K Flex Software on the instrument's controlling computer): Reaction Volume per Well (25 μL), Pre-Read Stage (60.0° for 30 seconds); Hold Stage (95.0° C. for 10 minutes); PCR Stage Step 1 (95.0° for 15 seconds); PCR Stage Step 2 (60.0° for 1 minute); Post-Read Stage (60.0° for 30 seconds); Number of Cycles (40); Auto Delta (Not enabled), and Starting Cycle (1).

Genotyping Procedure (B)—Sequencing:

The determination and evaluation of four (4) genetic variants of APOL1 (Kopp, J. B., et al., “APOL1 genetic variants in focal segmental glomerulosclerosis and HIV-associated nephropathy,” J Am Soc Nephrol., 2011, 22, 2129-2137 (describes nomenclature for G1/G2 alleles)) that are associated with FSGS by specific direct nucleotide sequencing using procedures to PCR amplify the genomic regions containing the APOL1 gene variants with gene specific primers is outline in Table 8:

TABLE 8

| Protein | rs#/ | | | Homozygous | Heterozygous |
|---|---|---|---|---|---|
| Variant | mutation | Gene | Allele | results | results |
| p.S342G | rs73885319 | APOL1 | G1 haplotype | A/A or G/G | A/G |
| | c.1024A > G | | | | |
| p.I384M | rs60910145 | APOL1 | G1 haplotype | T/T or G/G | T/G |
| | c.1152T > G | | | | |
| p.NYK388K | rs71785313 | APOL1 | G2 haplotype | (TTATAA/TTATAA) | +/− |
| | c.1164_1169delTTATAA | | | as +/+ or −/− | |

Additional reagents utilized to determine the four (4) genetic variants detailed in Table 8 includes:

Oligonucleotide Polymerase Chain Reaction (“PCR”) Primers (sequences given in 5′-3′ orientation), as noted in Table 9, prepared as 0.2 μM Scale HPLC Purified (eurofins MWG Operon Huntsville, Ala.):

TABLE 9

| PCR | SEQ. | | |
|---|---|---|---|
| Primer | ID No. | Oligonucleotide Sequence | Genotype Variant |
| APOL1F1 | 1 | 5′-CGC-CAG-GGT-TTT-CCC-AGT-CAC-GAA-CCA- | APOL1 G1 and G2 |
| | | ATC-TCA-GCT-GAA-AGC-3′ | |
| APOL 1R3 | 2 | 5′-GAG-CCA-CTA-TCG-ACT-ACG-GCA-TCA- | APOL1 G1 and G2 |
| | | ATA-TCT-CTC-CTG-GTG-GCT-G-3′ | |

Genomic DNA known to amplify the APOL1 regions of interest are utilized as PCR controls.

Platinum Taq Polymerase (Invitrogen, Catalog #10966-034 Carlsbad, Calif.).

50 mM Magnesium Chloride (MgCl₂) (Invitrogen, Catalog #Y02016 Carlsbad, Calif.).

10 mM Deoxyribonucleoside Triphosphates (“dNTPs”), PCR grade (Invitrogen, Catalog #18427-013 or equivalent Carlsbad, Calif.).

10× PCR Buffer (Invitrogen, Catalog #Y02028 Carlsbad, Calif.).

Nuclease Free-water (Ambion, Catalog #AM9932 or equivalent Carlsbad, Calif.).

Tris/EDTA (“TE”) buffer (10 mM Tris-HCl, 1.0 mM EDTA), pH 8.0 (Ambion, Catalog #AM9849 or equivalent Carlsbad, Calif.).

Ethidium Bromide Solution 10 mL (10 mg/mL) (Invitrogen Catalog #15585-011, or equivalent, Carlsbad , Calif.).

100 base pair (“bp”) DNA Ladder (Invitrogen #15628-019 Carlsbad, Calif.).

Latitude HT Precast Gel-2% agarose (Lonza #57246 or equivalent Hopkinton, Mass.).

Deionized water.

The following reagents are also utilized: 100 μM Oligonucleotide Primer Stocks, 10 μM Oligonucleotide Primer Stocks, 1× Tris Borate EDTA Running Buffer with 50 μg Ethidium Bromide, 1× Bromophenol Blue Agarose Gel Loading Dye Stock, 1× Agarose Gel Loading Dye, and 0.1 μg/μL 100 base pair DNA Ladder.

Equipment utilized: Eppendorf Mastercycler PCR instrument (Eppendorf).

Procedure: In general, target regions encompassing the three APOL1 variants representing APOL1 G1/G2 genotypes (r573885319, rs60910145, and r571785313) are PCR amplified using gene-specific oligonucleotide primer pairs shown in Table 9. As further detailed below, the resultant products are purified and subject to fluorescent di-deoxynucleotide DNA sequencing. The products are run on an ABI3730 48-capillary array for the collection of the sequence as electrophoretogram files. Electrophoretogram files for each individual's exons are exported and visualized using the bioinformatics software package Sequencher (GeneCodes Corporation) version 4.10.1. The gene sequence from each sample is compared to a consensus sequence to determine the genotype at APOL1 variant locations rs73885319, rs60910145, and rs71785313. Nucleotide numbering for APOL1 is based on NCBI reference sequence NM 003661.3, and NM_014625.2, respectively. All protein results are inferred based on nucleotide changes and are not verified experimentally. The procedure further involves the following steps:
1—A whole blood sample is collected (drawn) from a patient.
2—Process the whole blood sample for the isolation of DNA, producing two (2) DNA aliquots if possible.
3—Qualification of the oligonucleotide primer lots is verified.
4—Samples and controls are diluted to 5 ng/μL in nuclease-free water prior to analysis.
5—A no-template control (NTC) blank is included in the primary PCR for each variant assay, comprising nuclease-free water in place of any sample or positive control. The NTC is run once for each primary PCR setup.
6—For patient samples, genotype each of the four (4) variants in duplicate according to the following sampling plan: If two DNA aliquots are available, sample and analyze each aliquot once; if only one DNA aliquot is available, sample and analyze in duplicate.
7—A Plate Map is prepared and completed, indicating the positions of samples and controls.
8—Each of the following is thawed: 10× PCR Buffer, 50 mM MgCl₂, 10 mM dNTP, 10 μM forward and reverse primer (for the variant of interest) at room temperature. The Platinum Taq™ DNA polymerase is removed from the freezer and placed on ice.
11—The Mastermix(es) are prepared, and briefly vortexed to mix, in a pre-PCR hood using the following reagent volumes (provided as single reaction volumes/final concentrations): Nuclease free water (17.75 μL/−), 10× PCR Buffer 2.5 μL/1×); 50 mM MgCl₂ (0.75 μL/1.5 mM), 10 mM dNTP mix (0.25 μL/0.1 mM), 10 mM Forward Primer (0.5 μL/0.1 mM), 10 mM Reverse Primer (0.5 μL/0.1 mM), Platinum Taq Polymerase (0.25 μL/1.25 units); such that the final volume was 22.5 μL.
10—Aliquot 22.50 μL of mastermix is placed into each experimental well, the plate is sealed and moved to the designated sample addition hood, and then seal is removed.
11—Sample Addition: using a new pipet tip for each sample, pipet 2.50 μL of DNA (diluted to 5 ng/μL) into the appropriate well based on the completed Plate Map. Pipet 2.50 μL of nuclease-free water is added to the negative control well. Pipette up and down to mix.
12—Plate is sealed with PCR plate adhesive film and centrifuged at 1250±250g for 5-10 sec.
13—The plate is placed on the Eppendorf Mastercycler PCR instrument, lid is closed, and the appropriate “APOL1” protocol is selected from the available list of cycling programs having the following settings: Step 1, 94° C. for 2 min.; Step 2, (30 cycles): 94° C. for 20 sec., 58° C. for 30 sec., 72° C. for 30 sec.; Step 3, 72° C. for 4 min.; and Step 4, Hold at 4° C.
14—After the thermocycling program finishes, the plate is removed from the thermocycler and the PCR products of all samples and controls are analyzed by agarose gel electrophoresis.
15—Evaluation of Primary PCR Amplicons:
   i) a precast gel (Lonza) and plastic tray are removed from a sealed bag, transferred to a gel box, the gel is covered with a TruBand™ Anchor (aligning the wells with openings), and enough 1× TBE with Ethidium Bromide solution (·1L) is added to completely submerge the gel and tray but not the TruBand™ anchor;
   ii) for every sample analyzed, pipet 5 μL 1× bromophenol blue loading dye into each well of a Falcon 3911 U bottom 96 well tray, pipet 10 μL of each PCR sample into the wells with the loading dye and mixed by gently pipetting up and down 2 to 3 times;
   iii) load 3 μL of the prepared 100 bp DNA ladder (0.1 μg/μL stock) to at least one well in each row of the gel;
   iv) load the entire volume of each sample (or up to 12 μL, whichever is smaller) to the appropriate well on the agarose gel;
   v) slide TruBand™ anchor to cover the wells and the gel is run at approximately 180V for approximately 20 minutes or until bromophenol blue dye front migrates at least ⅔ of the way to the next row of wells or the bottom of the gel;
   vi) gel is removed from the gel box, photographed using the Alphainnotech Gel Imaging system (SCI-IM-195), producing two thermal paper images: (1) an image optimized for composition (zoom as necessary to fill frame) and exposure (adjust focus as necessary), and (2) a 3D image of the original image; saving each gel image file to the C:\ drive of a dedicated instrument computer;
   vii) the controls and samples on the gel image are assessed to determine whether the PCR was successful according to the acceptance criteria detailed in Table 10. The presence of amplified product in the negative control (NTC) results in a failed assay and requires immediate re-amplification of all samples and controls, and both replicates of a sample are required to generate a band of the expected size (any sample replicate that fails amplification requires re-amplification (in duplicate)).

TABLE 10
Primary PCR Control Acceptance Criteria

| | Appearance | APOL1 Amplicon Size | |
|---|---|---|---|
| Positive Control | A clearly visible band | 353 bp | 392 bp |
| (genomic DNA) | | | |
| NTC | No band, but primer-dimer | N/A | N/A |
| | may be visible | | |
| Samples | A clearly visible band | 353 bp | 392 bp |

16—Analysis of the Samples' PCR products by Sequencing: (1) each sample's PCR product is purified with Exo-SAP-IT purification reagents; (2) sequencing reactions for the samples' PCR products is performed as follows: each “Exosapped” product is amplified once in both forward and reverse reactions, using the volumes (one reaction volume)) noted in Table 11 to prepare the desired quantity of each master mix, and then the purified amplicons are sequenced and interpreted.

TABLE 11
Forward and Reverse Reaction Mixtures

| Reagent | Forward Reaction | Reverse Reaction |
|---|---|---|
| Big Dye Ready-Reaction Mix | 4 μL | 4 μL |
| Forward Primer (M13-47) | 0.8 μL | N/A |
| Reverse Primer (pBrBamHI) | N/A | 1.6 μL |
| Nuclease-free water | 2.7 μL | 1.9 μL |
| Final volume | 7.5 μL | 7.5 μL |

17—Data Analysis For APOL1 Sequencing:
   i) the AB1 sequence files from ABI 3730 run folder are imported into the Gene Codes Sequencher software;
   ii) the resulting forward and reverse sequence for each control sample are aligned to the reference sequence file;
   iii) the sequences for each sample's forward and reverse reaction are reviewed, and the variant sequence for each sample replicate is recorded as either a homozygous result or heterozygous result, as presented in Table 8 (in cases where one reaction (forward or reverse) yields a poor quality sequence, the other may be used to read the sequence, provided that sequence is of high quality). Both replicates of a test sample are required to yield identical sequence results. If results are inconsistent among replicates, the sample is re-evaluated, and if any replicate fails due to no, or poor, amplification, a result is not recorded until a confirmatory result is obtained.

APOL1 Genotyping:

One tube of whole blood was collected from each patient and extracted in duplicate to generate two aliquots of genomic DNA. As provided in Table 12, each aliquot was analyzed to determine each patient's APOL1 genotype using either Genotyping Procedure (A) or Procedure (B), as detailed above:

TABLE 12
Forward and Reverse Reaction Mixtures

| | | | | | Genotyping |
|---|---|---|---|---|---|
| Gene | Accesssion # | SNP ID | Change/Location | Risk Allele | Procedure |
| APOL1 | NM_003661.3 | rs73885319 | c.1024A > G; p.S342G | G1 | (A) Sequencing |
| | | rs60910145 | c.1152T > G; p.I384M | G1 | (A) Sequencing |
| | | rs71785313 | c.1164 1169delTTATAA; | G2 | (A) Sequencing |
| | | | p.Asn388_Tyr389del | | |
| | | rs2239785 | c.448G > A; p.Glu150Lys | c.448G | (B) Allelic |
| | | | | | Discrimination |

The APOL1 genotyping results for each of the treated patients is summarized in Tables 13-15 by treatment group and stratified by race and prior calcineurin inhibitors (CNI) therapy:

TABLE 13
Test Pharmaceutical Formulation at 1 mg/kg

| | | | FSGS | APOL1 | |
|---|---|---|---|---|---|
| | | Prior | histological | genotype | |
| Patient | Race | CNI? | Subtype | variant | APOL1 haplotype |
| 1A | B | Y | NOS | Yes | Homozygous G1 |
| 2A | NB | N | Collapsing | Yes | G1 carrier |
| 3A | NB | Y | NOS | negative | negative |
| 4A | B | N | Collapsing | n/a | n/a |
| 5A* | NB | N | NOS | negative | negative |
| 6A | NB | N | NOS | negative | negative |
| 7A | NB | N | Tip | negative | negative |
| 8A* | NB | N | Tip | negative | negative |
| 9A | NB | Y | N/A | negative | negative |
| 10A | NB | Y | NOS | negative | negative |
| 11A | NB | Y | NOS | negative | negative |
| 12A* | NB | Y | NOS | negative | negative |
| 13A | NB | Y | Perihilar | negative | negative |
| 14A | NB | Y | NOS | negative | negative |

Table Note:
*= received other immunosuppressant medications;
NB = Non-Black;
B = Black;
n/a—sample not available for genotyping.

TABLE 14
Test Pharmaceutical Formulation at 4 mg/kg

| | | | FSGS | APOL1 | |
|---|---|---|---|---|---|
| | | Prior | histological | genotype | |
| Patient | Race | CNI? | Subtype | variant | APOL1 haplotype |
| 1B | B | N | Collapsing | Yes | G1/G2 carrier |
| 2B | B | Y | Cellular | Yes | G2 carrier |
| 3B | NB | N | Tip | negative | negative |
| 4B* | NB | N | Collapsing | negative | negative |
| 5B | NB | Y | Collapsing | negative | negative |
| 6B* | NB | Y | Cellular | negative | negative |
| 7B* | NB | Y | NOS | negative | negative |
| 8B | NB | Y | NOS | negative | negative |
| 9B | NB | Y | Tip | negative | negative |
| 10B | NB | Y | Tip | negative | negative |
| 11B | NB | Y | NOS | negative | negative |
| 12B | NB | Y | NOS | negative | negative |

Table Note:
*= received other immunosuppressant medications;
NB = Non-Black;
B = Black.

TABLE 15
Placebo Formulation

| | | | FSGS | APOL1 | |
|---|---|---|---|---|---|
| | | Prior | histological | genotype | |
| Patient | Race | CNI? | Subtype | variant | APOL1 haplotype |
| 1C | B | Y | Collapsing | Yes | G1/G2 carrier |
| 2C | NB | N | NOS | negative | negative |
| 3C | B | Y | NOS | negative | negative |
| 4C | NB | Y | NOS | negative | negative |
| 5C | NB | Y | Tip | negative | negative |
| 6C* | NB | Y | NOS | negative | negative |
| 7C | NB | Y | NOS | negative | negative |
| 8C* | NB | Y | NOS | negative | negative |
| 9C | NB | Y | Perihilar | negative | negative |
| 10C | NB | Y | NOS, Tip | negative | negative |

Table Note:
*= received other immunosuppressant medications;
NB = Non-Black;
B = Black.

APOL1 G1 and G2 haplotypes, which have been associated with increased risk for FSGS in people of African descent, and were present in a total of 6 study subjects (5 black subjects and 1 non-Black Hispanic subject). The homozygous APOL1 G1 finding (G1/G1) is interpreted as “strongly associated with FSGS disease in the African-American population” while the APOL1 G1 carrier (G1/− or G1/G2) and/or APOL1 G2 carrier (G2/− or G1/G2) results are interpreted as “potential risk in the African-American population”. One non-Black, Hispanic patient (4016-0001) from the U.S. is an APOL-1 G1 carrier; it is not known if patient has any African ancestry.

Dosage and duration: Each of the 36 randomized patients received at least one study treatment, and all were included in the Safety Dataset. In the Placebo Formulation arm, 10 patients (100%) completed all 4 study treatments. In the Test Pharmaceutical Formulation at 1 mg/kg arm (n=14), 13 patients (93%) completed all 4 study infusions and 1 (7.1%) completed 3 infusions. In the Test Pharmaceutical Formulation at 4 mg/kg arm (n=12), 10 patients (83%) completed all 4 study treatments, 1 patient (8.3%) completed 3 study treatments, and 1 patient (8.3%) completed 2 study treatments.

Statistical methods: The efficacy analyses were performed using the Full Analysis (FA) Set (all randomized patients who were treated with at least 1 dose of study drug and had at least 1 post-baseline Up/c assessment). An analysis using a Per Protocol (PP) Set (all FAS evaluable patients with no significant deviations deemed to possibly influence the efficacy analysis), restricted to primary efficacy, was planned if the PPS was less than 80% of the FAS; no PPS analysis was performed, as the criterion was not met.

The continuous parameters of Up/C ratio and eGFR are provided below in Tables 16-51, summarized by treatment group (Tables 16-29: Test Pharmaceutical Formulation at 1 mg/kg; Tables 30-41: Test Pharmaceutical Formulation at 4 mg/kg; and Tables 52-51: Placebo Formulation). Study infusions were conducted at visits 2-5 (“V2-V5), and the follow-up period was conducted at vistis 6-9 (”V6-V9″). Immunosuppressant medications were allowed during the follow-up period. The average of the 2 first morning void measurements collected on 2 separate days for every visit, for each patient, was used in all efficacy analyses of the Up/C ratio. For each parameter, the comparison of percent change from baseline to Day 112/ET was conducted using a mixed model repeated measures approach. The mixed model repeated measures model included prior CNI therapy (yes, no), treatment group, race (black, non-black), and baseline Up/C ratio and baseline eGFR as covariates. The model was fit using the SAS PROC MIXED procedure and treatment differences at time points were estimated and tested from the model. Nonparametric analysis was also performed for percent change in Up/C ratio, eGFR, and urinary protein excretion rate to Day 112 using nonparametric ANCOVA.

TABLE 16
Test Pharmaceutical Formulation at 1 mg/kg - Patient 1A

| | | eGFR | Urinary Protein (mg)/ |
|---|---|---|---|
| Visit | Day | (mL/min/1.73 m²) | Urinary Creatinine (mg) |
| screen | screen | 68.1 | 4.398 |
| 2 | −3 to 1 | 75.3 | 5.7175 |
| 3 | 21-22 | 75.3 | 2.0245 |
| 4 | 41-42 | 75.3 | 3.974 |
| 5 | 63-64 | 68.1 | 2.464 |
| 6 | 105-106 | 61.5 | 3.0845 |
| 7 | 140-141 | 61.5 | 3.051 |
| 8 | 168-169 | 67.5 | 3.75 |
| 9 | 252-253 | 61.5 | 3.061 |

TABLE 17
Test Pharmaceutical Formulation at 1 mg/kg - Patient 2A

| | | eGFR | Urinary Protein (mg)/ |
|---|---|---|---|
| Visit | Day | (mL/min/1.73 m²) | Urinary Creatinine (mg) |
| screen | screen | 62.6 | 6.273 |
| 2 | −1 to 1 | 57.1 | 4.0515 |
| 3 | 29 | 52.4 | 4.2255 |
| 4 | 57 | 52.4 | 3.1435 |
| 5 | 78 | 44.9 | 1.8535 |
| 6 | 107 | 52.4 | 1.7985 |
| 7 | 141 | 52.4 | 1.5185 |
| 8 | 170 | 48.2 | 1.4845 |
| 9 | 253 | 44.8 | n/a |

Table Note:
n/a—sample not available.

TABLE 18
Test Pharmaceutical Formulation at 1 mg/kg - Patient 3A

| | | eGFR | Urinary Protein (mg)/ |
|---|---|---|---|
| Visit | Day | (mL/min/1.73 m²) | Urinary Creatinine (mg) |
| screen | screen | 45.5 | 5.529 |
| 2 | 1 | 45.5 | 5.9095 |
| 3 | 27 | 42.2 | 5.514 |
| 4 | 49-50 | 39.3 | 6.617 |
| 5 | 73-74 | 34.5 | 2.945 |
| 6 | 112-113 | 39.3 | 3.516 |
| 7 | 139-141 | 42.1 | 3.261 |
| 8 | 167-168 | 42.1 | 3.4005 |
| 9 | 253 | 42.1 | 0.943 |

TABLE 19
Test Pharmaceutical Formulation at 1 mg/kg - Patient 4A

| | | eGFR | Urinary Protein (mg)/ |
|---|---|---|---|
| Visit | Day | (mL/min/1.73 m²) | Urinary Creatinine (mg) |
| screen | screen | 31.6 | 17.3125 |
| 2 | 1 | n/a | n/a |
| 3 | 28-29 | 25.2 | 18.534 |
| 4 | 57 | 22.4 | 17.2965 |
| 5 | 85 | 25.2 | 10.392 |
| 6 | 113 | 22.4 | 13.0865 |
| 7 | 146 | 18.1 | 8.941 |
| 8 | 167 | 16.5 | 8.4035 |
| 9 | 253 | 17 | 4.4665 |

Table Note:
n/a—sample not available.

TABLE 20
Test Pharmaceutical Formulation at 1 mg/kg - Patient 5A*

| | | eGFR | Urinary Protein (mg)/ |
|---|---|---|---|
| Visit | Day | (mL/min/1.73 m²) | Urinary Creatinine (mg) |
| screen | screen | 34.6 | 5.024 |
| 2 | −1 to 1 | 48.2 | 4.0785 |
| 3 | 21-22 | 36.7 | 2.5155 |
| 4 | 43 | 41.8 | 3.146 |
| 5 | 64 | 39.1 | 6.992 |
| 6 | 98-99 | 44.8 | 7.1695 |
| 7 | 127 | 34.6 | 5.157 |
| 8 | 155 | 36.7 | 5.134 |
| 9 | 239 | 48.2 | 10.5905 |

Table Notes:
*= received other immunosuppressant medications.

TABLE 21
Test Pharmaceutical Formulation at 1 mg/kg - Patient 6A

| | | eGFR | Urinary Protein (mg)/ |
|---|---|---|---|
| Visit | Day | (mL/min/1.73 m²) | Urinary Creatinine (mg) |
| screen | screen | 98.4 | 4.3245 |
| 2 | 1 | 87.2 | 5.049 |
| 3 | 21-22 | 70.6 | 3.79 |
| 4 | 43 | 87.2 | 4.284 |
| 5 | 64 | 70.6 | n/a |
| 6 | 113 | 70.6 | 4.219 |
| 7 | 148 | 87.2 | 4.4225 |
| 8 | 182 | 78.1 | 4.703 |
| 9 | 250 | n/a | 5.661 |

Table Note:
n/a—sample not available.

TABLE 22
Test Pharmaceutical Formulation at 1 mg/kg - Patient 7A

| | | eGFR | Urinary Protein (mg)/ |
|---|---|---|---|
| Visit | Day | (mL/min/1.73 m²) | Urinary Creatinine (mg) |
| screen | screen | 33.6 | 4.3605 |
| 2 | 1 | 35.5 | 3.2195 |
| 3 | 27 | 37.6 | 3.8485 |
| 4 | 56 | 37.6 | 5.013 |
| 5 | 84 | 25 | 3.563 |
| 6 | 108 | 30.1 | 4.0575 |
| 7 | 136 | 35.4 | 5.2905 |
| 8 | 163 | 31.7 | 4.4225 |
| 9 | 252 | 27.4 | 2.821 |

TABLE 23
Test Pharmaceutical Formulation at 1 mg/kg - Patient 8A*

| | | eGFR | Urinary Protein (mg)/ |
|---|---|---|---|
| Visit | Day | (mL/min/1.73 m²) | Urinary Creatinine (mg) |
| screen | screen | 75 | 9.245 |
| 2 | 1 | 75 | 9.745 |
| 3 | 29 | 65.5 | 6.4715 |
| 4 | 63 | 52 | 8.6755 |
| 5 | 85 | 58 | 5.001 |
| 6 | 110-111 | 51.8 | 7.196 |
| 7 | 138-139 | 65.2 | 1.8955 |
| 8 | 166-167 | 65.2 | 2.3865 |
| 9 | 243-244 | 87.2 | 0.6305 |

Table Notes:
*= received other immunosuppressant medications.

TABLE 24
Test Pharmaceutical Formulation at 1 mg/kg - Patient 9A

| | | eGFR | Urinary Protein (mg)/ |
|---|---|---|---|
| Visit | Day | (mL/min/1.73 m²) | Urinary Creatinine (mg) |
| screen | screen | 83.1 | 8.0405 |
| 2 | 1 | n/a | n/a |
| 3 | 31 | 97 | 4.479 |
| 4 | 57 | 97 | 5.577 |
| 5 | 85 | 83.1 | 4.704 |
| 6 | 112-113 | 83.1 | 5.893 |
| 7 | 141 | 83.1 | 6.7305 |
| 8 | 176 | 96.4 | 4.0855 |
| 9 | 246 | 72.1 | 7.0255 |

Table Note:
n/a - sample not available.

TABLE 25
Test Pharmaceutical Formulation at 1 mg/kg - Patient 10A

| | | eGFR | Urinary Protein (mg)/ |
|---|---|---|---|
| Visit | Day | (mL/min/1.73 m²) | Urinary Creatinine (mg) |
| screen | screen | 53.6 | 2.885 |
| 2 | 1 | 70.6 | 2.649 |
| 3 | 29 | 70.6 | 2.6105 |
| 4 | 64 | 63.8 | 3.8665 |
| 5 | 92 | 70.6 | 2.43 |
| 6 | 120 | 70.6 | 3.108 |
| 7 | 149 | 63.8 | 3.3085 |
| 8 | 179 | 63.8 | 3.0185 |
| 9 | 247 | 58.2 | 3.049 |

TABLE 26
Test Pharmaceutical Formulation at 1 mg/kg - Patient 11A

| | | eGFR | Urinary Protein (mg)/ |
|---|---|---|---|
| Visit | Day | (mL/min/1.73 m²) | Urinary Creatinine (mg) |
| screen | screen | 63 | 5.9375 |
| 2 | 1 | 55.8 | n/a |
| 3 | 24 | 55.8 | 4.7895 |
| 4 | 52 | 63 | 3.4695 |
| 5 | 87 | 63 | 4.469 |
| 6 | 115 | 63 | 2.5755 |
| 7 | 148 | 72.2 | 5.648 |
| 8 | 171 | 63 | 4.0665 |
| 9 | 253 | 62.8 | 6.284 |

Table Note:
n/a - sample not available.

TABLE 27
Test Pharmaceutical Formulation at 1 mg/kg - Patient 12A*

| | | eGFR | Urinary Protein (mg)/ |
|---|---|---|---|
| Visit | Day | (mL/min/1.73 m²) | Urinary Creatinine (mg) |
| screen | screen | 41.5 | 5.209 |
| 2 | −1 to 1 | 34.4 | 9.5855 |
| 3 | 21-22 | 36.5 | 4.064 |
| 4 | 41-42 | 41.3 | 4.82 |
| 5 | 63-64 | 36.3 | 6.085 |
| 6 | 105-106 | 36.3 | 3.9825 |
| 7 | 140-141 | 32.4 | 4.094 |
| 8 | 168-169 | 38.7 | 2.4 |
| 9 | 252-253 | 34.3 | 6.0235 |

Table Notes:
*= received other immunosuppressant medications.

TABLE 28
Test Pharmaceutical Formulation at 1 mg/kg - Patient 13A

| | | eGFR | Urinary Protein (mg)/ |
|---|---|---|---|
| Visit | Day | (mL/min/1.73 m²) | Urinary Creatinine (mg) |
| screen | screen | 42 | 8.7455 |
| 2 | −1 to 1 | 42.0 | 6.347 |
| 3 | 21-22 | 42 | 8.106 |
| 4 | 44-45 | 42 | 5.6215 |
| 5 | 65-66 | 39 | 6.031 |
| 6 | 115-117 | 34 | 5.1645 |
| 7 | 145 | 32 | 3.7025 |
| 8 | 179-180 | 34 | 6.0625 |
| 9 | 249-250 | 34 | 3.968 |

TABLE 29
Test Pharmaceutical Formulation at 1 mg/kg - Patient 14A

| | | eGFR | Urinary Protein (mg)/ |
|---|---|---|---|
| Visit | Day | (mL/min/1.73 m²) | Urinary Creatinine (mg) |
| screen | screen | 102.8 | 9.227 |
| 2 | 1 | 25.8 | 14.24 |
| 3 | 21-22 | 102 | 12.5915 |
| 4 | 44 | 102 | 11.3275 |
| 5 | 64 | 87.4 | 12.415 |
| 6 | 113 | 67.6 | 10.236 |
| 7 | 141 | 87.4 | 8.0085 |
| 8 | 162 | 102 | 7.9435 |
| 9 | 261 | 87.4 | 7.6585 |

TABLE 30
Test Pharmaceutical Formulation at 4 mg/kg - Patient 1B

| | | eGFR | Urinary Protein (mg)/ |
|---|---|---|---|
| Visit | Day | (mL/min/1.73 m²) | Urinary Creatinine (mg) |
| screen | screen | 64.8 | 6.25 |
| 2 | 1 | 72.3 | 6.4145 |
| 3 | 23 | 64.8 | 7.1955 |
| 4 | 51-52 | 64.8 | 5.1565 |
| 5 | 86 | 64.8 | 3.3245 |
| 6 | 107 | 53.2 | 3.3125 |
| 7 | 149 | 58.4 | 3.405 |
| 8 | 176 | 53.2 | 2.664 |
| 9 | 288 | 48.9 | 2.2435 |

TABLE 31
Test Pharmaceutical Formulation at 4 mg/kg - Patient 2B

| | | eGFR | Urinary Protein (mg)/ |
|---|---|---|---|
| Visit | Day | (mL/min/1.73 m²) | Urinary Creatinine (mg) |
| screen | screen | 104.3 | 2.8635 |
| 2 | −4 to 1 | 93.4 | 1.3265 |
| 3 | 28 | 104.3 | 2.4185 |
| 4 | 62 | 134.9 | 1.4735 |
| 5 | 82-83 | 117.8 | 1.138 |
| 6 | 110-111 | 104.3 | 1.191 |
| 7 | 143-144 | 117.8 | 0.3415 |
| 8 | 166 | 117.8 | 1.0075 |
| 9 | 238 | 117.1 | 2.1195 |

TABLE 32
Test Pharmaceutical Formulation at 4 mg/kg - Patient 3B

| | | eGFR | Urinary Protein (mg)/ |
|---|---|---|---|
| Visit | Day | (mL/min/1.73 m²) | Urinary Creatinine (mg) |
| screen | screen | 71.1 | 9.5105 |
| 2 | −1 to 1 | 113.5 | 11.934 |
| 3 | 29 | 71.1 | 9.738 |
| 4 | 64 | 78.6 | 8.3215 |
| 5 | 102 | 50.7 | 9.284 |
| 6 | 112 | n/a | n/a |
| 7 | 140-141 | 70.6 | 7.9605 |
| 8 | 204 | 78.1 | 8.159 |
| 9 | 252 | n/a | n/a |

Table Note:
n/a - sample not available.

TABLE 33
Test Pharmaceutical Formulation at 4 mg/kg - Patient 4B*

| | | eGFR | Urinary Protein (mg)/ |
|---|---|---|---|
| Visit | Day | (mL/min/1.73 m²) | Urinary Creatinine (mg) |
| screen | screen | 62.8 | 2.9185 |
| 2 | −1 to 1 | 62.8 | 6.5015 |
| 3 | 26 | 62.8 | 6.9305 |
| 4 | 56-57 | 62.8 | 3.4765 |
| 5 | 85 | 62.8 | 8.7585 |
| 6 | 109-110 | 62.8 | 6.2955 |
| 7 | 145 | 58.0 | 7.7395 |
| 8 | 166 | 50.2 | 6.125 |
| 9 | 250 | 53.8 | 5.9635 |

Table Notes:
*= received other immunosuppressant medications.

TABLE 34
Test Pharmaceutical Formulation at 4 mg/kg - Patient 5B

| | | eGFR | Urinary Protein (mg)/ |
|---|---|---|---|
| Visit | Day | (mL/min/1.73 m²) | Urinary Creatinine (mg) |
| screen | screen | 51.0 | 7.4815 |
| 2 | −1 to 1 | 55.6 | 14.823 |
| 3 | 29 | 23.9 | 12.193 |
| 4 | 77-78 | 21.9 | 22.2955 |
| 5 | 116 | 23.8 | 30.3155 |
| 6 | 151 | 15.1 | 30.269 |
| 7 | 175 | 13.9 | 23.024 |
| 8 | 193 | 16.1 | 18.756 |
| 9 | 252 | 13.6 | 52.3065 |

TABLE 35
Test Pharmaceutical Formulation at 4 mg/kg - Patient 6B*

| | | eGFR | Urinary Protein (mg)/ |
|---|---|---|---|
| Visit | Day | (mL/min/1.73 m²) | Urinary Creatinine (mg) |
| screen | screen | 100.4 | 4.095 |
| 2 | 1 | 57.3 | 2.7205 |
| 3 | 36 | 100.4 | 3.158 |
| 4 | 57 | 87.6 | 5.949 |
| 5 | 78 | 48.6 | 7.2485 |
| 6 | 106 | 69.5 | 4.063 |
| 7 | 141 | 87.6 | 6.468 |
| 8 | 162 | 45.1 | 5.2095 |
| 9 | 255 | 48.6 | 7.205 |

Table Notes:
*= received other immunosuppressant medications.

TABLE 36
Test Pharmaceutical Formulation at 4 mg/kg - Patient 7B*

| | | eGFR | Urinary Protein (mg)/ |
|---|---|---|---|
| Visit | Day | (mL/min/1.73 m²) | Urinary Creatinine (mg) |
| screen | screen | 36.3 | 9.8315 |
| 2 | −2 to 1 | 19.0 | 15.8785 |
| 3 | 33-34 | 33.8 | 10.847 |
| 4 | 61-63 | 26.5 | 13.0155 |
| 5 | 82-83 | 25.1 | 19.815 |
| 6 | 103-106 | 20.7 | 23.609 |
| 7 | 139-140 | 26.4 | 19.291 |
| 8 | 167-169 | 29.6 | 20.8545 |
| 9 | 253-262 | 42.1 | 14.527 |

Table Notes:
*= received other immunosuppressant medications.

TABLE 37
Test Pharmaceutical Formulation at 4 mg/kg - Patient 8B

| | | eGFR | Urinary Protein (mg)/ |
|---|---|---|---|
| Visit | Day | (mL/min/1.73 m²) | Urinary Creatinine (mg) |
| screen | screen | 55.6 | 7.9345 |
| 2 | −1 to 1 | 45.8 | 9.6045 |
| 3 | 28 | 45.8 | 7.7135 |
| 4 | 49 | 45.8 | 12.2815 |
| 5 | 84 | 50.3 | 9.3465 |
| 6 | 111-112 | 50.3 | 8.6955 |
| 7 | 147 | 42.1 | 10.4975 |
| 8 | 181 | 50.0 | 15.1355 |
| 9 | 243 | 38.7 | 10.104 |

TABLE 38
Test Pharmaceutical Formulation at 4 mg/kg - Patient 9B

| | | eGFR | Urinary Protein (mg)/ |
|---|---|---|---|
| Visit | Day | (mL/min/1.73 m²) | Urinary Creatinine (mg) |
| screen | screen | 145.9 | 11.2615 |
| 2 | −1 to 1 | 145.9 | 10.009 |
| 3 | 30 | 145.9 | 8.245 |
| 4 | 58 | 188.7 | 9.612 |
| 5 | 85 | 145.9 | 11.01 |
| 6 | 119-120 | 145.9 | 8.4645 |
| 7 | 148 | 118.2 | 7.556 |
| 8 | 177 | 145.9 | 5.1355 |
| 9 | 254 | 145.9 | 7.093 |

TABLE 39
Test Pharmaceutical Formulation at 4 mg/kg - Patient 10B

| | | eGFR | Urinary Protein (mg)/ |
|---|---|---|---|
| Visit | Day | (mL/min/1.73 m²) | Urinary Creatinine (mg) |
| screen | screen | 94.2 | 13.341 |
| 2 | 1 | 112.5 | 4.8905 |
| 3 | 28 | 138.8 | 3.701 |
| 4 | 49 | 94.2 | 5.8435 |
| 5 | 70 | 112.5 | 3.354 |
| 6 | 112 | 112.5 | 2.871 |
| 7 | 140 | 111.9 | 4.5545 |
| 8 | 168 | 111.9 | 5.146 |
| 9 | 252 | 111.9 | 7.845 |

TABLE 40
Test Pharmaceutical Formulation at 4 mg/kg - Patient 11B

| | | eGFR | Urinary Protein (mg)/ |
|---|---|---|---|
| Visit | Day | (mL/min/1.73 m²) | Urinary Creatinine (mg) |
| screen | screen | 74.5 | 3.413 |
| 2 | −1 to 1 | 65.0 | 3.897 |
| 3 | 25 | 65.0 | 2.3985 |
| 4 | 46 | 65.0 | 3.1195 |
| 5 | 72 | 74.5 | 3.0365 |
| 6 | 112-114 | 65.0 | 3.785 |
| 7 | 143-144 | 64.8 | 2.5665 |
| 8 | 172 | 57.4 | 3.0255 |
| 9 | 249 | 64.8 | 2.432 |

TABLE 41
Test Pharmaceutical Formulation at 4 mg/kg - Patient 12B

| | | eGFR | Urinary Protein (mg)/ |
|---|---|---|---|
| Visit | Day | (mL/min/1.73 m²) | Urinary Creatinine (mg) |
| screen | screen | 61.1 | 5.716 |
| 2 | −1 to 1 | 39.5 | 3.2185 |
| 3 | 26-29 | 61.1 | 3.1255 |
| 4 | 56 | n/a | n/a |
| 5 | 70-71 | 60.8 | 3.8875 |
| 6 | 105-106 | 55.8 | 3.103 |
| 7 | 146-148 | 39.3 | 3.236 |
| 8 | 176 | 44.6 | 3.937 |
| 9 | 253 | 44.6 | 2.301 |

Table Note:
n/a—sample not available.

TABLE 42
Placebo Formulation - Patient 1C

| | | eGFR | Urinary Protein (mg)/ |
|---|---|---|---|
| Visit | Day | (mL/min/1.73 m²) | Urinary Creatinine (mg) |
| screen | screen | 87.9 | 3.417 |
| 2 | 1 | 87.9 | 4.557 |
| 3 | 25 | 77.9 | 3.8775 |
| 4 | 53 | 87.9 | 1.3055 |
| 5 | 81 | 87.9 | 3.9505 |
| 6 | 112 | 100.7 | 3.1835 |
| 7 | 137 | 87.9 | 3.5495 |
| 8 | 169 | 77.9 | 2.541 |
| 9 | 252 | 87.9 | 2.202 |

TABLE 43
Placebo Formulation - Patient 2C

| | | eGFR | Urinary Protein (mg)/ |
|---|---|---|---|
| Visit | Day | (mL/min/1.73 m²) | Urinary Creatinine (mg) |
| screen | screen | 32.7 | 4.635 |
| 2 | −2 to 1 | 37.0 | 6.7825 |
| 3 | 29 | 32.7 | 5.9885 |
| 4 | 57 | 34.7 | 3.571 |
| 5 | 92 | 32.6 | 4.2785 |
| 6 | 125 | 32.6 | 4.228 |
| 7 | 155 | 36.9 | 4.8965 |
| 8 | 182-183 | 30.9 | 3.2545 |
| 9 | 252-253 | 30.9 | 1.533 |

TABLE 44
Placebo Formulation - Patient 3C

| | | eGFR | Urinary Protein (mg)/ |
|---|---|---|---|
| Visit | Day | (mL/min/1.73 m²) | Urinary Creatinine (mg) |
| screen | screen | 65.7 | 4.898 |
| 2 | −3 to 1 | 65.7 | 7.526 |
| 3 | 28 | 59.4 | 13.5065 |
| 4 | 58 | 49.7 | 15.7265 |
| 5 | 90 | 42.6 | 15.2005 |
| 6 | 120 | 39.8 | 12.375 |
| 7 | 147 | 35.0 | 7.7435 |
| 8 | 176 | 39.8 | 8.8295 |
| 9 | 260 | 31.0 | 7.9405 |

TABLE 45
Placebo Formulation - Patient 4C

| | | eGFR | Urinary Protein (mg)/ |
|---|---|---|---|
| Visit | Day | (mL/min/1.73 m²) | Urinary Creatinine (mg) |
| screen | screen | 98.3 | 11.137 |
| 2 | −1 to 1 | 82.3 | 13.656 |
| 3 | 74 | 98.0 | 9.251 |
| 4 | 96 | 48.7 | 4.8915 |
| 5 | 118 | 34.0 | 12.862 |
| 6 | 129-131 | 29.5 | 7.9365 |
| 7 | 145 | 36.9 | 9.7075 |
| 8 | 168 | 19.8 | 13.4125 |
| 9 | 308 | 24.4 | 29.163 |

TABLE 46
Placebo Formulation - Patient 5C

| | | eGFR | Urinary Protein (mg)/ |
|---|---|---|---|
| Visit | Day | (mL/min/1.73 m²) | Urinary Creatinine (mg) |
| screen | screen | 41.8 | 10.2885 |
| 2 | 1 | 47.6 | 9.117 |
| 3 | 22 | 36.8 | 7.474 |
| 4 | 51 | 47.6 | 13.245 |
| 5 | 85 | 39.1 | 7.821 |
| 6 | 113 | 36.8 | 7.962 |
| 7 | 148 | 34.8 | 7.6685 |
| 8 | 176 | 31.3 | 8.6525 |
| 9 | 261 | 39.1 | 9.361 |

TABLE 47
Placebo Formulation - Patient 6C*

| | | eGFR | Urinary Protein (mg)/ |
|---|---|---|---|
| Visit | Day | (mL/min/1.73 m²) | Urinary Creatinine (mg) |
| screen | screen | 61.8 | 7.6255 |
| 2 | 1 | 61.8 | n/a |
| 3 | 22 | 61.8 | n/a |
| 4 | 37-43 | 61.8 | 10.2555 |
| 5 | 64 | 61.8 | n/a |
| 6 | 111-113 | 61.8 | 7.916 |
| 7 | 141-144 | 25.3 | 6.6765 |
| 8 | 163-169 | 44.3 | 8.95 |
| 9 | 252-253 | n/a | 8.5305 |

Table Note:
*= received other immunosuppressant medications;
n/a—sample not available.

TABLE 48
Placebo Formulation - Patient 7C

| | | eGFR | Urinary Protein (mg)/ |
|---|---|---|---|
| Visit | Day | (mL/min/1.73 m²) | Urinary Creatinine (mg) |
| screen | screen | 44.7 | 3.8425 |
| 2 | 1 | 41.5 | 2.1925 |
| 3 | 23 | 41.5 | 4.4145 |
| 4 | 48 | 41.5 | 2.7455 |
| 5 | 71 | 36.2 | 1.9305 |
| 6 | 113 | 41.5 | 2.546 |
| 7 | 141 | 44.7 | 2.381 |
| 8 | 168 | 44.7 | 2.019 |
| 9 | 253 | 44.3 | 1.5565 |

TABLE 49
Placebo Formulation - Patient 8C*

| | | eGFR | Urinary Protein (mg)/ |
|---|---|---|---|
| Visit | Day | (mL/min/1.73 m²) | Urinary Creatinine (mg) |
| screen | screen | 159.3 | 3.222 |
| 2 | −3 to 1 | 196.6 | 4.6785 |
| 3 | 21-22 | 196.6 | 2.857 |
| 4 | 41-42 | 196.6 | 4.938 |
| 5 | 63-64 | 196.6 | 4.1925 |
| 6 | 105-106 | 196.6 | 6.537 |
| 7 | 143 | 159.3 | 2.4965 |
| 8 | 176 | 159.3 | n/a |
| 9 | 252-253 | 158.2 | 6.081 |

Table Note:
*= received other immunosuppressant medications;
n/a—sample not available.

TABLE 50
Placebo Formulation - Patient 9C

| | | eGFR | Urinary Protein (mg)/ |
|---|---|---|---|
| Visit | Day | (mL/min/1.73 m²) | Urinary Creatinine (mg) |
| screen | screen | 58.4 | 6.1735 |
| 2 | 1 | 58.4 | 5.444 |
| 3 | 43 | 58.4 | 6.5165 |
| 4 | 70 | 50.0 | 6.56 |
| 5 | 91 | 46.6 | 4.9485 |
| 6 | 115-116 | 50.0 | 5.122 |
| 7 | 144 | 50.0 | 6.2935 |
| 8 | 168-169 | 43.4 | 6.318 |
| 9 | 252-253 | 40.8 | 5.3895 |

TABLE 51
Placebo Formulation - Patient 10C

| | | eGFR | Urinary Protein (mg)/ |
|---|---|---|---|
| Visit | Day | (mL/min/1.73 m²) | Urinary Creatinine (mg) |
| screen | screen | 47.9 | 6.211 |
| 2 | −1 to 1 | 47.9 | 6.033 |
| 3 | 23 | 47.9 | 5.3535 |
| 4 | 44 | 47.9 | 4.6265 |
| 5 | 65 | 47.7 | 5.2225 |
| 6 | 111-112 | 38.7 | 8.7485 |
| 7 | 134-135 | 25.3 | 8.5555 |
| 8 | 177 | 27.8 | 9.2985 |
| 9 | 240 | 16.3 | 8.923 |

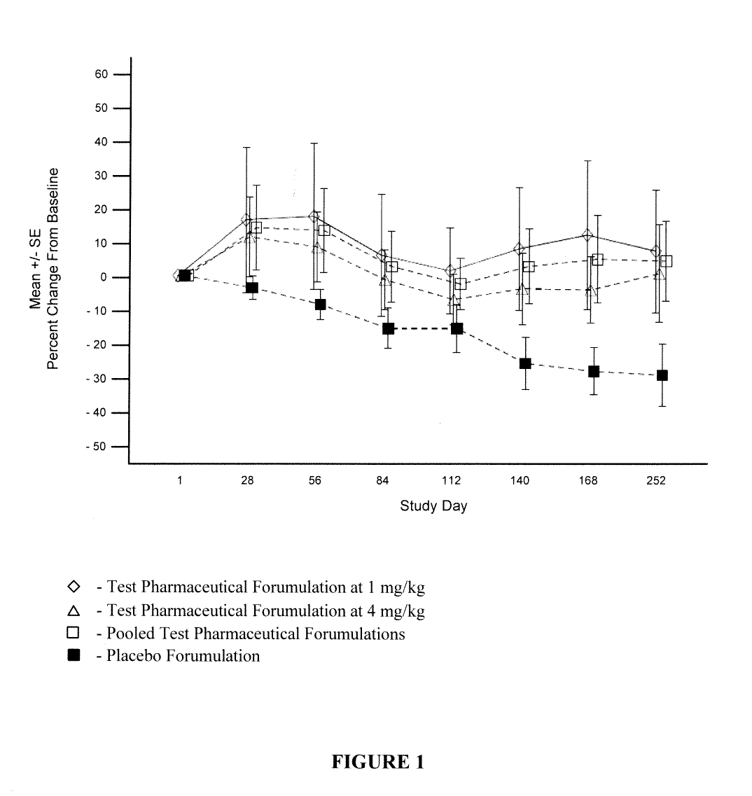

A general trend towards stabilization of mean eGFR (mL/min per 1.73 m²) over time, up through study day 252 (or up through visit 9) was observed in the actively treated groups (Test Pharmaceutical Formulation at 1 mg/kg and Test Pharmaceutical Formulation at 4 mg/kg), whereas the mean eGFR levels (mL/min per 1.73 m²) in the placebo group (treated with Placebo Formulation) progressively declined over time. These general trends are illustrated in FIG. 1, and the number of patients included in the grouped data points at each particular study day are provided in Table 52.

TABLE 52
Number of Patients evaluated for Percent
Change in mean eGFR (mL/min per 1.73 m²)

| | Number of Patients in Treatment Group | | | | | | | |
|---|---|---|---|---|---|---|---|---|
| | Study Day | | | | | | | |
| | 1 | 28 | 56 | 84 | 112 | 140 | 168 | 252 |
| Test Pharmaceutical | 14 | 14 | 14 | 14 | 14 | 14 | 14 | 14 |
| Formulation at 1 mg/kg | | | | | | | | |
| Test Pharmaceutical | 12 | 12 | 12 | 12 | 12 | 11 | 11 | 11 |
| Formulation at 4 mg/kg | | | | | | | | |
| Pooled Test | 26 | 26 | 26 | 26 | 26 | 25 | 25 | 25 |
| Pharmaceutical | | | | | | | | |
| Formulations | | | | | | | | |
| Placebo Formulation | 10 | 10 | 10 | 10 | 10 | 10 | 10 | 9 |

All publications and patent applications mentioned in this specification are herein incorporated by reference in their entirety to the same extent as if each individual publication or patent application was specifically and individually indicated to be incorporated by reference in their entirety.

While preferred embodiments of the present invention have been shown and described herein, it will be obvious to those skilled in the art that such embodiments are provided by way of example only. It is intended that the following claims define the scope of the invention and that methods and structures within the scope of these claims and their equivalents be covered thereby.

1. A method of treating primary focal segmental glomerulosclerosis (primary FSGS) in a patient having an APOL1 variant, comprising administering a therapeutically effective amount of TGFβ antagonist to the patient.

2. The method of claim 1, wherein the TGFβ antagonist is administered intravenously to the patient.

3-4. (canceled)

5. The method of claim 1, wherein the method stabilizes the patient's glomerular filtration rate.

6. (canceled)

7. The method of claim 1, wherein the TGFβ antagonist is a pan-specific TGFβ antagonist and neutralizes active isoforms of TGFβ.

8. The method of claim 7, wherein the pan-specific TGFβ antagonist is fresolimumab.

9-10. (canceled)

11. The method of claim 8, wherein 1-4 mg/kg of the fresolimumab is intravenously administered to the patient, based on the total weight of said patient.

12. The method of claim 11, wherein the 1-4 mg/kg of the fresolimumab is intravenously administered monthly, every 28 days, every 21 days, or every 14 days to said patient.

13-22. (canceled)

23. The method of claim 12, wherein the fresolimumab is provided as a lyophilized powder and is reconstituted in sterile water for injection (sWFI) prior to intravenous administration, wherein the reconstituted fresolimumab composition is a phosphate-buffered solution containing 10 mg/mL fresolimumab, 30 mg/mL mannitol, 10 mg/mL sucrose, 0.1 mg/mL polysorbate 80, and 1.5 mg/mL sodium chloride.

24-32. (canceled)

33. The method of claim 12, wherein the fresolimumab is administered to the patient in 1-10 treatment sessions.

34. (canceled)

35. The method of claim 8, wherein the fresolimumab is administered in a sufficient dose to achieve a trough serum plasma level of fresolimumab of 103 to 51,209 ng/mL.

36. (canceled)

37. The method of claim 1, wherein the treated patient has a 50% or greater decline in Up/C ratio (mg protein/mg creatinine) from baseline.

38. The method of claim 1, wherein the treated patient has a decline in Up/C ratio from baseline to a level in the range of from ≥0.3 to ≤3.0 mg protein/mg creatinine

39. The method of claim 1, wherein the treated patient has a decline in Up/C ratio from baseline to a level of <0.3 mg protein/mg creatinine.

40. The method of claim 1, wherein the APOL1 variant is an APOL1 G1 haplotype, APOL-1 G2 haplotype, or a diplotype having both G1 and G2 haplotypes.

41. The method of claim 40, wherein the variant patient is homozygous positive for APOL1 G1 haplotype (G1/G1) or is a heterozygous carrier for APOL1 G1 haplotype comprising G1/− or diplotype G1/G2.

42. The method of claim 40, wherein the the patient is homozygous positive for APOL1 G2 haplotype (G2/G2) or is a potential heterozygous carrier for APOL1 G2 haplotype comprising G2/− or diplotype G1/G2.

43-45. (canceled)

46. The method of claim 1, wherein the method further comprises genotyping a blood sample from the patient prior to treatment.

47-48. (canceled)

49. The method of claim 1, wherein the patient prior to treatment is diagnosed as having one or more of the following conditions:
nephrotic syndrome,
nephrotic proteinuria,
an eGFR of 30 mL/min/1.73m² or more,
a Up/C ratio of 3 mg protein/mg creatinine or more in at least one urinary sample collected,
a Up/C of 2 mg protein/mg creatinine or more in at least two urinary samples collected,
resistance to steroid therapy, and
intolerance to steroid therapy.

50-56. (canceled)

57. The method of claim 1, wherein the patient prior to treatment is intolerant or resistant to high-dose steroid therapy for at least 4 weeks.

58. The method of claim 1, wherein the patient prior to treatment has been treated with an angiotensin-converting enzyme inhibitor (ACEI), an angiotensin II receptor blocker (ARB), or both, at a stable dose for at least 4 weeks.

59-76. (canceled)
